(19) United States
(12) Patent Application Publication
(10) Pub. No.: US 2006/0263792 A1
(43) Pub. Date: Nov. 23, 2006

(54) Use of CR1-binding molecules in clearance and induction of immune responses

(75) Inventors: Nehal Mohamed, Budd Lake, NJ (US); George L. Spitalny, Morristown, NJ (US); Leslie S. Casey, New York, NY (US); Steven Marion Jones, Denville, NJ (US); Steven E. Pincus, Hackettstown, NJ (US)

Correspondence Address: LAHIVE & COCKFIELD, 28 STATE STREET, BOSTON, MA, 02109, US

(73) Assignee: EluSys Therapeutics, Inc., Pine Brook, NJ (US)

(21) Appl. No.: 11/263,569

(22) Filed: Oct. 31, 2005

Related U.S. Application Data
(60) Provisional application No. 60/623,736, filed on Oct. 29, 2004.
(60) Provisional application No. 60/664,472, filed on Mar. 22, 2005.
(60) Provisional application No. 60/720,789, filed on Sep. 26, 2005.
(60) Provisional application No. 60/720,956, filed on Sep. 26, 2005.

(51) Int. Cl. C12Q 1/68; G01N 33/53

(52) U.S. Cl. 435/6; 435/7.1

(57) ABSTRACT

The present invention provides methods and compositions related to the discovery of molecules capable of both inducing an immune response to an antigen in a mammal and also effecting clearance of the antigen, with such molecules comprising a first moiety comprising an antigen binding portion which binds specifically to complement receptor 1 (CR1) and does not substantially bind to complement receptor 2 (CR2), linked to a second moiety which comprises the antigen or binds to the antigen. Methods of producing such molecules and their therapeutic and/or prophylactic uses are also featured.

### RELATED APPLICATIONS

This application claims priority to U.S. Ser. No. 60/623,736, filed on Oct. 29, 2004, titled “BISPECIFIC MOLECULES THAT BIND TO PROTEIN A AND METHODS OF THEIR USE”; U.S. Ser. No. 60/664,472, filed on Mar. 22, 2005, titled “BISPECIFIC MOLECULES THAT BIND TO PROTEIN A AND METHODS OF THEIR USE”; U.S. Ser. No. 60/720,789, filed on Sep. 26, 2005, titled “USE OF CR1-SPECIFIC MOLECULES IN CLEARANCE AND INDUCTION OF IMMUNE RESPONSES”; and U.S. Ser. No. 60/720,956, filed on Sep. 26, 2005, titled “CONSTRUCTS THAT SPECIFICALLY RECOGNIZE CR1 AND THEIR USE TO INDUCE IMMUNE RESPONSES”. This application is also related to U.S. Ser. No. 10/812,636, filed on Mar. 29, 2004, titled “METHODS AND COMPOSITIONS FOR CONVERSION OF ANTIBODY ACTIVITY.” The entire contents of each of these applications are hereby incorporated herein by reference.

### BACKGROUND OF THE INVENTION

Primate erythrocytes, or red blood cells (RBC's), play an essential role in the clearance of antigens from the circulatory system. The formation of an immune complex in the circulatory system activates the complement factor C3b in primates and leads to the binding of C3b to the immune complex. The C3b/immune complex then binds to the type 1 complement receptor (CR1), a C3b receptor, expressed on the surface of erythrocytes via the C3b molecule attached to the immune complex. The immune complex is then chaperoned by the erythrocyte to the reticuloendothelial system (RES) in the liver and spleen for neutralization. The RES cells, most notably the fixed-tissue macrophages in the liver called Kupffer cells, recognize the C3b/immune complex and break this complex from the RBC by severing the C3b receptor-RBC junction, producing a liberated erythrocyte and a C3b/immune complex which is then engulfed by the Kupffer cells and is completely destroyed within subcellular organelles of the Kupffer cells. This pathogen clearance process, however, is complement-dependent, i.e., confined to immune complexes recognized by the C3b receptor, and is ineffective in removing immune complexes which are not recognized by the C3b receptor.

Taylor et al. have discovered a complement independent method of removing pathogens from the circulatory system. Taylor et al. have shown that chemical crosslinking of a first monoclonal antibody (mAb) specific for a primate C3b receptor to a second monoclonal antibody specific to a pathogenic antigenic molecule creates a bispecific heteropolymeric antibody or bispecific heteropolymeric (HP) which binds a pathogenic antigenic molecule to a primate's C3b receptor without complement activation (U.S. Pat. Nos. 5,487,890; 5,470,570; and 5,879,679). Taylor also reported an HP which can be used to remove a pathogenic autoantibody from the circulation. Such an HP, also referred to as an “Antigen-based Heteropolymer” (AHP), contains an antibody that recognizes CR1 cross-linked to an antigen recognized by a pathogenic autoantibody (see, e.g., U.S. Pat. No. 5,879,679; Lindorfer et al., 2001, Immunol Rev. 183: 10-24; Lindorfer et al., 2001, J. Immunol Methods 248: 125-138; Ferguson et al., 1995, Arthritis Rheum 38: 190-200). HPs and AHPs have not been shown to induce immune responses.

Complement receptor 2 (CR2) is a mammalian cell surface receptor whose sequence and evolutionary origin in mammals is related to complement receptor 1 (CR1). In mice, CR1 and CR2 arise from alternative splice variants of the CR2 gene, while in humans, CR1 and CR2 are encoded by separate loci located within 75 kb of one another in the genome, suggesting that a duplication event occurred in this region sometime after murine and human evolutionary lineages diverged. Human CR1 and CR2 proteins share only 34% sequence identity (though certain domains, particularly repeat domains of the two proteins, share greater than 70% identity), are expressed on distinct cell types, and show differing complement-binding specificities. CR1 is present on B and some T lymphocytes, Follicular dendritic cells, neutrophils, monocytes, eosinophils, mast cells, glomerular podocytes, and erythrocytes, whereas CR2 is expressed on B cells, follicular dendritic cells, and possibly pharyngeal epithelial cells. While CR1 binds to C3b with highest affinity (also binding to iC3b, C4b and C3 in which the internal thioester has been hydrolyzed), CR2 binds to C3dg, C3d, iC3b, and weakly, C3b. Targeting of antigens to CR2 has recently been shown to provoke an immune response to the CR2-targeted antigen. However, no such effect has been described for antigens specifically targeted to CR1.

Developing compositions and methods to reduce infection and/or to reduce virulence in animals (e.g., mammals, harboring pathogens) e.g., due to toxins, infectious pathogens or opportunistic organisms and/or unwanted cells comprising an antigenic marker(s) represents a significant challenge. The discovery of new compositions and methods for clearing a selected antigen from the circulation and/or inducing immune responses to a selected antigen would allow for development of improved prophylactics and therapeutics, including vaccines and anti-pathogen, toxin and cancer treatments. Methods of clearing an antigen and/or inducing immune responses to such antigens would allow for greater efficacy in treating and/or preventing infection or disease in a subject would be of tremendous benefit.

### SUMMARY OF THE INVENTION

The present invention advances the art by providing compositions and methods for clearing molecules from a subject and/or induction of immune responses.

In one embodiment, the instant methods employ constructs which are effective for clearance of an antigen from the circulation and/or from tissues of a subject, the construct comprising a first moiety comprising a moiety that binds to CR1 linked to a second moiety which binds to an antigen. In another embodiment, such a construct is effective in inducing and/or enhancing an immune response.

In other embodiments, the invention features compositions that specifically bind mammalian CR1 capable of inducing and/or enhancing immune responses in a subject, while not effecting clearance of the antigen from the circulation of the subject.

In one embodiment, a construct of the invention comprises a moiety that binds CR1 and does not substantially bind CR2 and a moiety comprising an antigen or a molecule that binds to an antigen.

In one aspect, the invention is directed to a method for inducing an immune response to an antigen in a mammal comprising administering a molecule effective for clearance of the antigen from the circulation, wherein the molecule comprises a first moiety which binds specifically to complement receptor 1 (CR1) linked to a second moiety which binds to the antigen. In one embodiment, the first moiety comprises an antibody. In a related embodiment, the antibody is an anti-human CR1 antibody, optionally selected from the group consisting of 7G9, H4, E11, H9, and YZ-1. In another embodiment, at least one of the first or second moiety comprises an antibody or an antigen binding portion thereof. In certain embodiments, at least one of the first or second moiety is selected from the group consisting of a Fab fragment, a F(ab′)₂ fragment, a single chain antibody, and an scFv molecule.

In an additional embodiment, the antigen is a pathogenic agent or epitope derived therefrom. In a related embodiment, the pathogenic agent is selected from the group consisting of a virus, a bacterium, a fungus, and a parasite or an epitope derived therefrom. In a further embodiment, the pathogenic agent binds to a receptor on a host cell and the second moiety comprises a soluble form of the receptor. In another embodiment, the pathogenic agent is a virus and the second moiety comprises a soluble form of a cellular receptor that binds to the virus. In certain embodiments, the second moiety is a small molecule or a drug. In a specific embodiment, the pathogenic agent is a fungus and the second moiety comprises amphotericin B. In another embodiment, the antigen is a toxin or an epitope derived therefrom. In certain embodiments, the antigen is selected from the group consisting of a tumor cell, a tumor cell toxin, an epitope derived from a tumor cell, and an epitope derived from a tumor cell toxin. In a further embodiment, the antigen is a pathogenic protein. In other embodiments, the epitope is selected from the group consisting of a protein, a peptide, a carbohydrate, a lipid, a lipopolysaccharide, a polysaccharide, a small molecule, glycoprotein, and a peptidoglycan.

In one embodiment, the first and second moieties are linked via a chemical crosslinker. In certain embodiments, the chemical crosslinker comprises polyethelyene glycol (PEG) as a spacer. In another embodiment, the first and second moieties are covalently linked. In a further embodiment, the first and second moieties are non-covalently linked. In a specific embodiment, the first and second moieties are linked via a genetic fusion.

In certain embodiments, the molecule is a heteropolymer. In another embodiment, the molecule is a bispecific antibody. In a further embodiment, the molecule is a fusion protein. In a specific embodiment, the antigen comprises a non-infectious form of a pathogen, a vaccine strain of a pathogen, or epitope derived therefrom, and the method further comprises administering the antigen to the mammal. In one related embodiment, the antigen is administered prior to the molecule. In another related embodiment, the antigen is administered with the molecule. In a further related embodiment, the antigen is administered after the molecule. In certain embodiments, the antigen is part of the construct.

In one embodiment, at least the first or the second moiety of the molecule is a human antibody. In certain embodiments, at least the first or the second moiety of the molecule is modified to decrease immunogenicity. In a specific embodiment, at least one of the first or the second moiety of the molecule comprises an entity selected from the group consisting of a chimeric antibody or antigen binding portion thereof, a humanized antibody or antigen binding portion thereof, and a deimmunized antibody or antigen binding portion thereof.

In certain embodiments, the immune response is a protective immune response against the antigen. In one embodiment, a disease is treated in the mammal. In another embodiment, a disease is prevented in the mammal. In a further embodiment, an infection is treated in the mammal. In certain embodiments, the infection is a bacterial infection. In other embodiments, the infection is a viral infection. In additional embodiments, the infection is a fungal infection. In one embodiment, the infection is a parasitic infection. In another embodiment, the infection is nosocomial. In some embodiments, the infection is prevented in the mammal. In certain embodiments, the mammal is at risk for recurring infections. In a related embodiment, the mammal has had recurring infections. In an additional embodiment, the molecule is administered prior to an invasive medical procedure. In a specific embodiment, the procedure is a surgical procedure.

In another aspect, the invention features a composition for inducing an immune response to an antigen in a subject, comprising administering a molecule effective for clearance of the antigen from the circulation, wherein the molecule comprises a first moiety which binds specifically to human complement receptor 1 (CR1) linked to a second moiety which binds to the antigen. A related aspect features a molecule comprising a first moiety which binds to complement receptor 1 (CR1), linked to a second moiety which binds to Staphylococcus aureus protein A, wherein the molecule is effective for clearance of the antigen from the circulation.

In one embodiment, the second moiety comprises an antibody or an antigen binding portion thereof. In certain embodiments, the second moiety comprises an antibody fragment selected from the group consisting of a Fab, a F(ab′)₂, a single chain antibody, an scFv molecule, and a protein A binding portion of an Fc molecule. In a specific embodiment, the second moiety comprises an anti-protein A antibody or antigen binding portion thereof. In another embodiment, administration of a molecule of the invention to a subject is used to prevent a bacterial infection in the subject.

In an additional aspect, the invention features a method of inducing clearance of an antigen from the circulation, comprising administering to a mammal having an antigen in its circulation a molecule that comprises a first moiety that specifically binds to CR1 and does not substantially bind to CR2 and a second moiety which binds to the antigen.

In a further aspect, the invention is directed to a construct for inducing an immune response to an antigen in a mammal comprising a first moiety which specifically binds to complement receptor 1 (CR1) linked to a second moiety which binds to the antigen, wherein the construct is not effective for clearing the antigen from the circulation. In a related aspect, the invention is directed to a construct for inducing an immune response to an antigen in a mammal comprising a first moiety which specifically binds to complement receptor 1 (CR 1) linked to a second moiety which comprises the antigen to which an immune response is desired.

In one embodiment, the first moiety comprises an antibody. In certain embodiments, the antibody is an anti-human CR1 antibody. In specific embodiments, the antibody is selected from the group consisting of 7G9, H4, E11, H9, and YZ-1. In another embodiment, the antigen is a vaccine strain of a pathogen. In an additional embodiment, the first moiety comprises C3b or C4b.

In certain embodiments, the second moiety comprises an antibody or an antigen binding portion thereof. In a related embodiment, the antibody or antigen binding portion thereof is bound to the antigen. In specific embodiments, at least one of the first or second moiety is selected from the group consisting of a Fab fragment, a F(ab′)₂ fragment, a single chain antibody, and an scFv molecule.

In another embodiment, the antigen is a pathogenic agent or epitope derived therefrom. In certain embodiments, the pathogenic agent is selected from the group consisting of a virus, a bacterium, a fungus, and a parasite or an epitope derived therefrom. In a related embodiment, the pathogenic agent has a cellular receptor and the second moiety comprises a soluble form of the cellular receptor. In an additional embodiment, the pathogenic agent is a virus and the second moiety comprises a soluble form of a cellular receptor that binds to a virus.

In one embodiment, the second moiety is a small molecule or a drug. In another embodiment, the pathogenic agent is a fungus and the second moiety comprises amphotericin B.

In an additional embodiment, the antigen is a toxin or an epitope derived therefrom. In certain embodiments, the antigen is selected from the group consisting of a tumor cell, a tumor cell toxin, an epitope derived from a tumor cell, and an epitope derived from a tumor cell toxin. In specific embodiments, the epitope is selected from the group consisting of a protein, a peptide, a carbohydrate, a lipid, a lipopolysaccharide, a polysaccharide, a small molecule, a glycoprotein, and a peptidoglycan. In another embodiment, the antigen is a pathogenic protein. In a further embodiment, the antigen comprises a portion of an antibody that generates anti-idiotypic antibodies.

In some embodiments, the first and second moieties are linked via a chemical crosslinker. In certain embodiments, the chemical crosslinker comprises polyethelyene glycol (PEG) as a spacer. In another embodiment, the first and second moieties are covalently linked. In other embodiments, the first and second moieties are non-covalently linked. In an additional embodiment, the first and second moieties are linked via a genetic fusion. In a further embodiment, the first and second moieties are linked via a receptor-ligand interaction.

In certain embodiments, at least the first or the second moiety of the construct comprises a human antibody or antigen binding portion thereof. In a related embodiment, at least the first or the second moiety of the construct is modified to decrease its immunogenicity. In specific embodiments, at least one of the first or the second moiety of the construct comprises an entity selected from the group consisting of a human antibody or antigen binding portion thereof, a chimeric antibody or antigen binding portion thereof, a humanized antibody or antigen binding portion thereof, and a deimmunized antibody or antigen binding portion thereof.

In another aspect, the invention is directed to a method for inducing an immune response to an antigen in a mammal, comprising administering a construct comprising a first moiety which specifically binds to complement receptor 1 (CR1) linked to a second moiety which comprises the antigen to which an immune response is desired to a subject. In a related aspect, the invention features a method for inducing an immune response to an antigen in a mammal comprising administering a construct which is not effective for clearing the antigen from the circulation, the construct comprising a first moiety which specifically binds to complement receptor 1 (CR1) linked to a second moiety which binds to the antigen to a subject wherein an immune response to the antigen is induced in the mammal.

In one embodiment, the antigen comprises a non-infectious form of a pathogen, a vaccine strain of a pathogen, or epitope derived therefrom, the method further comprising administering the antigen to the mammal. In certain embodiments, the antigen is administered prior to the construct. In other embodiments, the antigen is administered with the construct. In specfic embodiments, the antigen is part of the construct. In another embodiment, the antigen is administered after the construct.

In an additional aspect, the invention is directed to a method for clearing an antigen from a tissue of a mammal via administration of a molecule effective for clearance of the antigen from the tissue, wherein the molecule comprises a first moiety which binds specifically to complement receptor 1 (CR1) linked to a second moiety which binds to the antigen, wherein the antigen is cleared from the tissue of the mammal. In certain embodiments, the tissue is an organ. In one embodiment, the organ is lung, liver or spleen.

### BRIEF DESCRIPTION OF THE FIGURES

FIG. 1 shows the protective effect on survival of anti-S. aureus Protein A HP administration at doses of 1 μg, 2 μg, 4 μg, 8 μg and 16 μg per mouse. Monoclonal S. Aureus Protein A antibody (MAb) at a dose of 50 μg per mouse and PBS were non-protective.

FIG. 2 depicts the therapeutic effect on survival of anti-Protein A HP administration at six hours after challenge with S. aureus.

FIG. 3 shows the persistent protective effect on survival of anti-S. aureus Protein A HP administration. All mice survived when HP administration was performed 30-45 minutes prior to first challenge with S. aureus strain MW2 or 13301, and mice were then challenged a second time 28 days later with either S. aureus strain MW2 or 13301.

FIG. 4 depicts that the protective effect on survival of anti-S. aureus Protein A HP administration also protects from a second challenge at 28 days with S. epidermidis.

FIG. 5 shows that the second challenge protective effect on survival of anti-S. aureus Protein A HP administration is likely due to an antibody response against both S. aureus and S. epidermidis.

FIG. 6 shows that administration of an anti-protein A HP construct cleared S. Aureus from the blood and organs (liver, kidney and spleen).

FIG. 7 shows that HPs provided protection against a lethal C. albicans challenge.

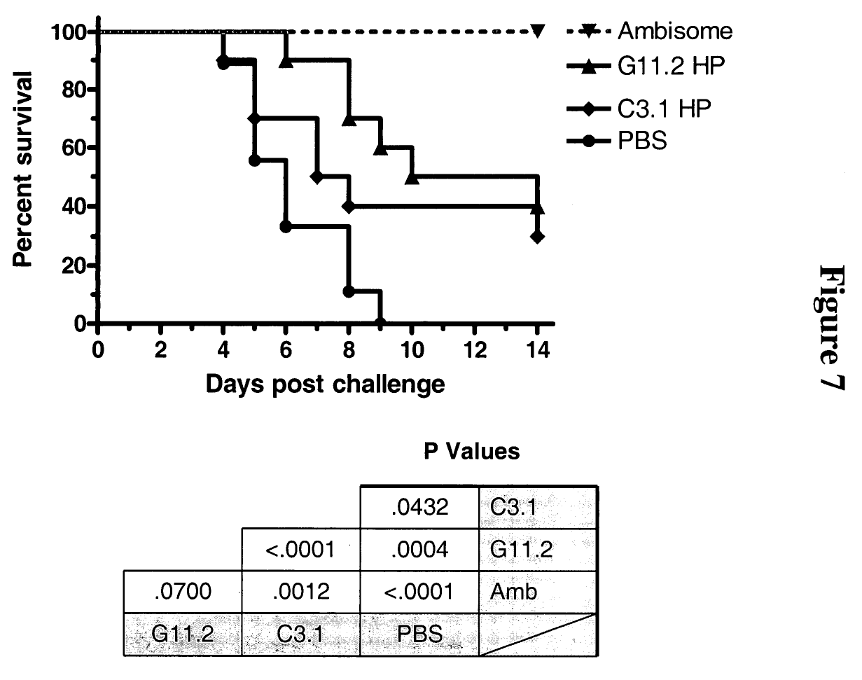

### DETAILED DESCRIPTION OF THE INVENTION

The present invention is based, at least in part, on the discovery of compositions that comprise a moiety that binds mammalian CR1 (e.g., a moiety that specifically binds to CR1 and does not substantially bind CR2) and an antigen or a moiety that binds to an antigen, mediate clearance of the antigen from a subject and/or induce or enhance an immune response in a subject. The present invention features, e.g., constructs, methods of producing the constructs of the invention as well as methods of therapeutic and/or prophylactic use of the constructs of the invention. In a specific embodiment, the present invention provides molecules comprising a first moiety comprising an antigen binding portion which binds to a cell surface receptor which mediates reticulendothelial cell clearance linked to a second moiety comprising an antigen binding portion which binds to a Staphylococcal surface antigen, e.g., Protein A. In one embodiment, a construct of the invention mediates clearance of an antigen from the circulation of a subject. In another embodiment, a construct of the invention mediates clearance of an antigen from the tissues of a subject. In one embodiment, such a construct induces an immune response in a subject. Additional embodiments feature compositions that specifically bind mammalian CR1 and induce and/or enhance immune responses to an antigen, while not effecting clearance of the antigen and methods of its use.

### I. DEFINITIONS

As used herein, the term “immune response” includes T cell mediated and/or B cell mediated immune responses. Exemplary immune responses include T cell responses, e.g., cytokine production, and cellular cytotoxicity. In addition, the term immune response includes antibody production (humoral responses) and activation of cytokine responsive cells, e.g., macrophages.

The term “antibody” as used herein refers to immunoglobulin molecules. The term “antibody” includes complete antibody molecules as well as antigen binding portions thereof. Immunoglobulin molecules are encoded by genes which include the kappa, lambda, alpha, gamma, delta, epsilon and mu constant regions, as well as a myriad of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (C_{H}1), hinge, constant heavy 2 (C_{H}2), and constant heavy 3 (C_{H}3) domains. The IgG heavy chains are further sub-classified based on their sequence variation, and the subclasses are designated IgG1, IgG2, IgG3 and IgG4. The term “antibody” includes, e.g., naturally occurring antibody or immunoglobulin molecules or modified (e.g., genetically engineered) antibody molecules that resemble naturally occurring antibody molecules. The term “antibody” as used herein also includes modified forms of antibody molecules, e.g., scfv molecules, minibodies, and the like. An antibody of the invention can belong to any one of these classes and/or isotypes. In one embodiment of the invention an antibody of the invention is non-neutralizing. In another embodiment, an antibody of the invention is neutralizing.

The term “antigen-binding portion” or “antigen binding fragment” of an antibody, as used herein, refers to one or more fragments of an antibody that retain the ability to specifically bind to an antigen (e.g., CR1). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such molecules are encompassed within the term “antigen-binding portion” of an antibody. These antibody fragments are obtained using conventional techniques known to those with skill in the art, and the fragments are screened for binding in the same manner as are intact antibodies.

A “chimeric” protein comprises a first amino acid sequence linked to a second amino acid sequence with which it is not naturally linked in nature. The amino acid sequences may normally exist in separate proteins that are brought together in the fusion polypeptide or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

The term “chimeric antibody”, as used herein, refers to a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, each of which is incorporated herein by reference in its entirety).

The term “humanized antibody”, as used herein, refers to an antibody molecule from non-human species having one or more complementarity determining regions (CDRs) from the non-human species and a framework region from a human immunoglobulin molecule. (see e.g., U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Natl. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

A deimmunized antibody that binds a human CR1 receptor and not CR2 can also be used in the present invention. As used herein, the term “deimmunized antibody” refers to an antibody that is of a non-human origin but has been modified, i.e., with one or more amino acid substitutions, so that it is non-immunogenic or less immunogenic to a human when compared to the starting non-human antibody. In preferred embodiments, the deimmunized anti-CR1 antibody comprises one or more non-human V_{H} or V_{L} sequences modified to comprise one or more amino acid substitutions so that the deimmunized antibody is non-immunogenic or less immunogenic to a human when compared to the respective unmodified non-human sequences (see WO 00/34317, WO 98/52976, and W02005/002529, all of which are incorporated herein by reference in their entirety).

As used herein, the term “crosslinking” refers to the covalent linkage of two proteins, generally via a non-peptide bond. Crosslinking agents can covalently react with sites on proteins or modified proteins to effect crosslinking. As used herein, the term “crosslinking agent” or “crosslinker” refers to a compound that is capable of covalently binding two molecules together. After the reaction, the crosslinker, or part of the crosslinker, generally forms a part of the linkage between the conjugated molecules.

With regard to the binding of an antibody to an antigen, the term “specific binding” or “specifically binds to” or is “specific for” a particular polypeptide or an epitope on a particular target means binding that is measurably different from a non-specific interaction. Preferably, any binding in the non-specific interaction is not substantially different from background. In one embodiment, the term “specific binding” refers to binding to a particular polypeptide or epitope on the molecule for which it is specific without substantial binding (e.g., exhibiting essentially background binding) to a molecule for which it is not specific. Specific binding can be measured, for example, by determining binding of a molecule compared to binding of a control molecule. Antibodies that exhibit “specific binding” or “specifically bind to” or are “specific for” a particular polypeptide or an epitope on a particular polypeptide target may have a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater.

For example the phrase, “specifically binds to complement receptor 1 (CR1)” refers to a moiety (e.g., antibody, C3b, C4b, etc.) that binds to CR1 and does not substantially bind to non-CR1 molecules (e.g., does not substantially bind to CR2). Such a moiety therefore binds CR1 with sufficient affinity such that the antibody molecule is useful as a prophylactic and/or therapeutic agent that binds to CR1 but does not significantly cross-react with CR2. In such embodiments, the extent of binding of the antibody to CR1 is at least about 5 or at least about 100 times background signal or noise, or more, as determined by, e.g., fluorescence activated cell sorting (FACS) analysis or radioimmunoprecipitation (RIA). In the same embodiments, any binding of the CR1-specific moiety to CR2 is effectively within the limits of background binding in such binding assays.

The term “clearing the antigen from the circulation” as used herein refers to the process by which compositions/complexes that bind to CR1 (e.g., bispecific heteropolymers comprising anti-CR1 antibody linked to antibodies that bind to pathogen) are removed from the circulation of a subject. While not wishing to be bound by theory, it is believed that clearance of such compositions is mediated, at least in part, via binding of such compositions to complement receptors of erythrocytes, resulting in delivery of the composition to the reticuloendothelial system (RES) in the liver and spleen, thereby removing the composition and the antigen to which the antibody in the composition binds from the circulation. The RES cells, most notably the fixed-tissue macrophages in the liver called Kupffer cells, likely break such compositions/complexes from the RBC, producing a liberated erythrocyte and a composition/complex which is then engulfed by the Kupffer cells and is completely destroyed within subcellular organelles of the Kupffer cells. A construct that is “effective for clearing the antigen from the circulation” is one that results in the clearance or removal of antigen from the circulation, e.g., by the above-described mechanism. In one embodiment, such a construct binds to Fcγ receptors on cells sufficiently to induce clearance. Such a construct may, for example, at least one (e.g., one or two) intact Fc region of an antibody.

In contrast, a construct that is “not effective for clearing the antigen from the circulation” is one that does not result in the clearance or removal of antigen from the circulation, e.g., by the above-described mechanism. In one embodiment, such a construct does not bind to Fcγ receptors on cells sufficiently to induce clearance. Such a construct may, for example, comprise one or more antibodies that have been modified using techniques known in the art to reduce or eliminate Fcγ receptor binding or comprise one or more antigen binding portions of an antibody which lack an Fc region of an antibody.

As used herein, the term “subject” includes a human or nonhuman mammal.

As used herein, the term “antigen presenting cell (APC)” refers to a class of immune cells capable of internalizing and processing an antigen, so that antigenic determinants are presented on the surface of the cell as MHC-associated complexes, in a manner capable of being recognized by the immune system (e.g., MHC class I restricted cytotoxic T lymphocytes and/or MHC class 11 restricted helper T lymphocytes). The two requisite properties that allow a cell to function as an APC are the ability to process endocytosed antigens and the expression of MHC gene products. Examples of APCs include dendritic cells (DC), mononuclear phagocytes (e.g., macrophages), B lymphocytes, Langerhans cells of the skin and, in humans, endothelial cells.

The term “antigen” or “immunogen” is used interchangeably and refers to a substance or a material that is specifically recognized by an antibody and to which an antibody can be generated. The antigen can be a whole molecule or a portion of a molecule, e.g., an epitope, against which an immune response is desired. Preferably, the term “antigen” as used here includes molecules or epitopes derived therefrom that are not widely expressed in the subject to be treated, e.g., non-self antigens or epitopes from pathogenic agents (such as viruses, bacteria, fungi, parasites), from pathogenic proteins (e.g., pathogenic amyoid or prion proteins) or that are tumor cell specific (e.g., a tumor cell, tumor cell toxin or epitope).

The term “epitope” includes antigenic determinants capable of specific binding to an antibody. Epitopes usually consist of chemically active surface groupings of molecules such as amino acids or sugar side chains and usually have specific three dimensional structural characteristics, as well as specific charge characteristics. Epitopes may be, e.g., protein, peptide, carbohydrate, lipid, lipopolysaccharide, polysaccharide, small molecule, glycoprotein, or peptidoglycan in nature.

As used herein, the term “vaccine strain of a pathogen” refers to a strain of a pathogen that is suitable for use in a vaccine. Vaccine strains pose less risk of serious consequences than disease causing strains of pathogens yet allow development of an immune response against the pathogenic strain. A “vaccine strain” can include, but is not necessarily limited to, a non-pathogenic strain, a killed strain (e.g., heat-killed, chemically-killed, irradiated or otherwise), an attenuated strain, or a strain that has been genetically modified to reduce its infectivity and/or virulence.

The terms “C3b” and “C4b” as used herein have their art-recognized meanings. These complement proteins bind specificity to CR1 and do not substantially bind to CR2.

As used herein, the term “C3b-like receptor” refers to a mammalian molecule expressed on the surface of a mammalian blood cell, which has an analogous function to primate CR1, in that it binds to C3b.

As used herein the term “pathogen” or “pathogenic agent” includes microorganisms that are capable of infecting or parasitizing normal hosts (e.g., animals (such as mammals, preferably primates, e.g. humans)). As used herein the term also includes microorganisms whose replication is unwanted in a subject or toxic molecules (e.g., toxins) produced by microorganisms. As used herein, the term opportunistic agents includes, e.g., microorganisms that are capable of infecting or parasitizing abnormal hosts, e.g., hosts in which normal flora have been supplanted, e.g., as a result of a treatment regimen, or immunocompromised hosts.

The term “small molecule”, as used herein, refers to a molecule which has a molecular weight of less than about 1 kD and most preferably less than about 0.4 kD. Examples of small molecules include, but are not limited to nucleotides, amino acids, peptides, peptidomimetics, carbohydrates, lipids or other organic (carbon containing) molecules. Organic small molecules typically have multiple carbon-carbon bonds.

The term “toxin”, as used herein, refers to agents made by eukaryotic cells, microbial cells, or synthetically produced which are capable of cytotoxicity against a host cell, a pathogen, or both. The term “toxin” includes agents capable of cellular toxicity, including for example anthrax toxin from B. anthracis, ricin from jack bean, diphtheria toxin, and other naturally-occurring (e.g., pathogen-derived) and man-made toxins. Tumor cell toxins are also examples of toxins.

The term “pathogenic protein” as used herein refers to one or more endogenous or exongenous proteins that are associated with a disease state or condition, for example a particular cancer, bacterial or viral infection. In general, such proteins have a direct or indirect pathogenic effect on eukaryotic cells. Exemplary pathogenic proteins include pathogenic forms of amyloid protein or prion proteins. The term also includes pathogenic fragments of such proteins.

As used herein, the term “genetic fusion” refers to a co-linear, covalent linkage of two or more proteins or fragments thereof via their individual peptide backbones, through genetic expression of a polynucleotide molecule encoding those proteins.

The term “receptor” or “cellular receptor” includes molecules capable of specifically binding to a ligand by affinity-based interactions that do not involve complementary base pairing. A ligand and its corresponding receptor are referred to herein as members of a specific binding pair (thus, the terms “ligand-receptor interaction” or “receptor-ligand interaction”, used interchangeably herein, refer to any specific binding of receptor and ligand moieties). “Cellular receptors” include receptors which are expressed on the surface of cells. The term “ligand” includes molecules capable of specifically binding to a receptor by affinity-based attraction.

The term “drug” as used herein includes a molecule, group of molecules, complex, or substance administered to an organism for diagnostic, therapeutic, medical or veterinary purposes.

The term “spacer molecule” or “spacer” refers to one or more molecules, groups or compounds selected or designed to join two molecules and preferably to alter or adjust the distance between the two molecules.

### II. CONSTRUCTS OF THE INVENTION

In one embodiment, the constructs of the invention comprise a first moiety that specifically binds to CR1 and a second moiety that either comprises an antigen or binds to an antigen.

In one embodiment, the first moiety binds to CR1 and does not substantially bind to CR2.

The constructs of the instant invention can be made using any combination of the moieties described herein. For example, exemplary constructs may include a CR1-binding moiety selected from the group consisting of: (a) anti-CR1-monoclonal antibodies (e.g., anti-CR1 specific monoclonal antibodies) or (b) an anti-CR1 antigen binding fragment, linked to a second moiety selected from the group consisting of: (a) an anti-antigen monoclonal antibody; (b) an anti-antigen scFv molecule; (c) an antigen binding fragment of an anti-pathogen antibody; (d) a vaccine strain of a pathogen, (e) an antigen or epitope thereof, (f) a toxin; (g) an antigenic and/or antigen-binding small molecule or drug; or (h) a receptor, ligand or other protein that binds to an antigen. Preferred constructs include, e.g.,: an anti-pathogen monoclonal antibody which does not bind to FcγRs or antigen binding portion thereof conjugated to an anti-CR1 specific scFv molecule; an anti-pathogen scFv molecule conjugated to an anti-CR1 specific scFv molecule; a vaccine strain of a pathogen conjugated to an anti-CR1 specific antibody or antigen binding portion thereof; a vaccine strain of a pathogen conjugated to an anti-specific CR1 antibody that does not bind to FcγRs or antigen binding portion thereof; an epitope of a pathogen conjugated to an anti-CR1 specific antibody that does not bind to FcγRs or antigen binding portion thereof; a vaccine strain of a pathogen conjugated to an anti-CR1 specific scFv molecule.

These and other exemplary moieties for use in the constructs of the invention are described in more detail below.

A. Moieties That Bind to CR1

Preferably, moieties that bind to CR1 do so specifically and do not substantially bind, e.g., to CR2.

1. CR1 Ligands: C3b and C4b

C3b and C4b are glycoproteins, and may be purified or isolated via genetic and/or organic means of synthesis. Native C3b and C4b are synthesized from C3 and C4, respectively. The proteins C3 and C4 contain an intramolecular thioester bond that not only controls their conformational state, and their ligand binding properties, but also mediates their covalent attachment to target nucleophiles on pathogen surfaces in a proteolytic activation-dependent manner. Whereas mature plasma C3 is a disulfide-linked heterodimer consisting of a 119-kDa -chain and a 75-kDa β-chain, plasma C4 is a disulfide-linked heterotrimer made up of a 93-kDa -chain, a 75-kDa β-chain, and a 33-kDa-chain. In both cases, proteolytic removal of a 77-residue activation peptide from the NH₂-terminal of the respective chains, i.e., C3a and C4a, respectively, results in exposure and activation of the thioester. Following thioester transacylation, or the competing hydrolysis reaction, the resulting C3b and C4b molecules acquire ligand-binding properties, including CR1-specificity, that were not present in the respective native molecules.

C3b and C4b each bind specifically to CR1. Accordingly, in one embodiment, C3b or C4b, or the CR1 binding portion thereof can be included in a construct of the invention to impart specific binding to CR1. C3 and C4 have similar overall structure, though the mature form of C4 (e.g., C4b) is processed into three chains, while C3b comprises two chains. Binding of CR1 receptors to C4b and C3b molecules involves repeat sequences within the CR1 receptor.

In one embodiment, a portion of a CR1 binding molecule may be included in a construct of the invention. CR1 binds to a region of C3b that is contained within the NH2 terminus of the alpha chain. A peptide from the NH2-terminal alpha chain fragment of C3c (X42, 42 residues in length from the NH2 terminus) was shown to inhibit binding of CR1 to C3b. In one embodiment, a construct of the invention may consist of a portion of the NH2 terminus or such a peptide. Becherer. 1988. J. Biol. Chem. 263:14586-91.

2. Antibodies that bind to CR1

In one embodiment of the invention, CR1 binding can be imparted by an antibody or antigen binding portion of an antibody that binds to CR1, e.g., that specifically binds to CR1 and does not substantially bind to CR2. An anti-CR1 antibody of the invention can be a novel antibody or an antibody that is known in the art to bind to CR1. The anti-CR1 antibodies of the invention bind specifically to CR1 and do not substantially bind to CR2. In one embodiment, such antibodies can be made using art-recognized methods, e.g., as described below.

a. Production of CR1 Antibodies

Exemplary antibodies may be obtained from natural sources or produced by hybridoma, recombinant or chemical synthetic methods, including modification of constant region functions by genetic engineering techniques (U.S. Pat. No. 5,624,821). The antibody of the present invention may be derived from a mammal and can be of any isotype.

An anti-CR1 mAb that specifically binds human CR1 can be produced using techniques know to one of ordinary skill in the art. For example, a mammal can be immunized with CR1 or a fragment thereof (or a highly homologous form of the molecule).

CR1 is a glycoprotein composed of a single polypeptide chain. Four allotypic forms of CR1 have been found, differing by increments of ˜40,000-50,000 daltons molecular weight. The two most common forms, the F and S allotypes, also termed the A and B allotypes, have molecular weights of 250,000 and 290,000 daltons (Dykman, T. R., et al., 1983, Proc. Natl. Acad. Sci. U.S.A. 80:1698; Wong, W. W., et al., 1983, J. Clin. Invest. 72:685), respectively, and two rarer forms have molecular weights of 210,000 and >290,000 daltons (Dykman, T. R., et al., 1984, J. Exp. Med. 159:691; Dykman, T. R., et al., 1985, J. Immunol. 134:1787). All four CR1 allotypes have C3b-binding activity (Dykman, T. R., et al., 1983, Proc. Natl. Acad. Sci. U.S.A. 80:1698; Wong, W. W., et al., 1983, J. Clin. Invest. 72:685; Dykman, T. R., et al., 1984, J. Exp. Med. 159:691; Dykman T. R., et al., 1985, J. Immunol. 134:1787).

At an appropriate time after immunization of the mammal e.g., when the specific antibody titers are highest, antibody-producing cells can be obtained from the subject and used to prepare monoclonal antibodies by standard techniques, such as the hybridoma technique originally described by Kohler and Milstein (1975, Nature 256:495-497), the human B cell hybridoma technique by Kozbor et al. (1983, Immunol. Today 4:72), the EBV-hybridoma technique by Cole et al. (1985, Monoclonal Antibodies and Cancer Therapy, Alan R. Liss, Inc., pp. 77-96) or trioma techniques. The technology for producing hybridomas is well known (see Current Protocols in Immunology, 1994, John Wiley & Sons, Inc., New York, N.Y.). Hybridoma cells producing a monoclonal antibody of the invention are detected by screening the hybridoma culture supernatants for antibodies that bind the polypeptide of interest and do not bind non CR1 molecules, e.g., CR2, e.g., using a standard ELISA.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. Monoclonal antibodies of the invention may also be made by recombinant DNA methods (U.S. Pat. No. 4,816,567).

In the hybridoma method of generating monoclonal antibodies, mammal, e.g., a mouse or a hamster, is immunized, e.g., described as hereinabove described to elicit lymphocytes that produce or are capable of producing antibodies that will bind to CR1 (see, e.g., U.S. Pat. No. 5,914,112, which is incorporated herein by reference in its entirety.)

Alternatively, lymphocytes may be immunized in vitro. Lymphocytes are then fused with myeloma cells using a suitable fusing agent, such as polyethylene glycol, to form a hybridoma cell (Goding, Monoclonal Antibodies: Principles and Practice, pp. 59 103, Academic Press, 1986). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT deficient cells.

Preferred myeloma cells are those that fuse efficiently, support stable high level production of antibody by the selected antibody producing cells, and are sensitive to a medium such as HAT medium. Among these, preferred myeloma cell lines are murine myeloma lines, such as those derived from MOPC 21 and MPC 11 mouse tumors available from the Salk Institute Cell Distribution Center, San Diego, Calif. USA, and SP 2 cells available from the American Type Culture Collection, Rockville, Md. USA. Human myeloma and mouse-human heteromyeloma cell lines also have been described for the production of human monoclonal antibodies (Kozbor, 1984, J. Immunol., 133:3001; Brodeur et al., Monoclonal Antibody Production Techniques and Applications, pp. 51 63 (Marcel Dekker, Inc., New York, 1987)). Culture medium in which hybridoma cells are growing is assayed for production of monoclonal antibodies directed against the antigen.

Preferably, the binding specificity of monoclonal antibodies produced by hybridoma cells is determined by immunoprecipitation or by an in vitro binding assay, such as radioimmunoassay (RIA) or enzyme linked immuno-absorbent assay (ELISA). The binding affinity of the monoclonal antibody can, for example, be determined by the Scatchard analysis of Munson et al., 1980, Anal. Biochem., 107:220.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59 103 (Academic Press, 1986)). Suitable culture media for this purpose include, for example, D-MEM or RPMI 1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A Sepharose, hydroxylapatite chromatography, gel electrophoresis, dialysis, or affinity chromatography.

Alternative to preparing monoclonal antibody-secreting hybridomas, an anti-CR1-specific antibody that does not bind CR2 can be identified using other art recognized techniques, e.g., can be isolated by screening a recombinant combinatorial immunoglobulin library (e.g., an antibody phage display library), e.g., with human CR1. Kits for generating and screening phage display libraries are commercially available (e.g., Pharmacia Recombinant Phage Antibody System, Catalog No. 27-9400-01; and the Stratagene antigen SurfZAP™ Phage Display Kit, Catalog No. 240612). Additionally, examples of methods and reagents particularly amenable for use in generating and screening antibody display library can be found in, for example, U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734.

In other embodiments, nucleic acid molecules encoding the heavy and light chains of an anti-CR1 mAb, preferably an anti-CR1 IgG, are prepared from the hybridoma cell line by standard methods known in the art. As a non-limiting example, cDNAs encoding the heavy and light chains of the anti-CR1 IgG are prepared by priming mRNA using appropriate primers, followed by PCR amplification using appropriate forward and reverse primers. Commercially available kits for cDNA synthesis can be used. The nucleic acids are used in the construction of expression vector(s). The expression vector(s) are transfected into a suitable host. Non-limiting examples include E. coli, yeast, insect cell, and mammalian systems, such as a Chinese hamster ovary cell line. Antibody production can be induced by standard method known in the art.

In embodiments where non-human antibodies or antigen binding portions thereof are incorporated into a construct, the antibody or antigen binding portion thereof may be modified to reduce its immunogenicity in a human subject. For example, techniques developed for the production of “chimeric antibodies” (Morrison et al., 1984, Proc. Natl. Acad. Sci., 81, 6851-6855; Neuberger et al., 1984, Nature 312, 604-608; Takeda et al., 1985, Nature, 314, 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, each of which is incorporated herein by reference in its entirety)

Humanized antibodies or antigen binding portions thereof can also be used in the constructs of the invention. Humanized antibodies are antibody molecules from non human species having one or more complementarity determining regions (CDRs) from the non human species and a framework region from a human immunoglobulin molecule. (see e.g., U.S. Pat. No. 5,585,089, which is incorporated herein by reference in its entirety.) Such chimeric and humanized monoclonal 5 antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. Nos. 4,816,567 and 5,225,539; European Patent Application 125,023; Better et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Canc. Res. 47:999-1005; Wood et al., 1985, Nature 314:446-449; Shaw et al., 1988, J. Nat]. Cancer Inst. 80:1553-1559; Morrison 1985, Science 229:1202-1207; Oi et al., 1986, Bio/Techniques 4:214; Jones et al., 1986, Nature 321:552-525; Verhoeyan et al., 1988, Science 239:1534; and Beidler et al., 1988, J. Immunol. 141:4053-4060.

Complementarity determining region (CDR) grafting is another method of humanizing antibodies. It involves reshaping murine antibodies in order to transfer full antigen specificity and binding affinity to a human framework (Winter et al. U.S. Pat. No. 5,225,539). CDR grafted antibodies have been successfully constructed against various antigens, for example, antibodies against IL 2 receptor as described in Queen et al., 1989 (Proc. Natl. Acad. Sci. USA 86:10029); antibodies against cell surface receptors CAMPATH as described in Riechmann et al. (1988, Nature, 332:323; antibodies against hepatitis B in Cole et al. (1991, Proc. Natl. Acad. Sci. USA 88:2869); as well as against viral antigens respiratory syncitial virus in Tempest et al. (1991, Bio Technology 9:267). CDR grafted antibodies are generated in which the CDRs of the murine monoclonal antibody are grafted into a human antibody. Following grafting, in one embodiment, additional amino acid changes in the framework region may be made to maintain affinity, presumably because framework residues are necessary to maintain CDR conformation, and some framework residues have been demonstrated to be part of the antigen binding site. However, in order to preserve the framework region so as not to introduce any antigenic site, the sequence is compared with established germline sequences followed by computer modeling.

A deimmunized antibody or antigen binding portion thereof can also be used in the present invention. As used herein, the term “deimmunized antibody” refers to an antibody that is of a non-human origin but has been modified, i.e., with one or more amino acid substitutions, so that it is non-immunogenic or less immunogenic to a human when compared to the starting non-human antibody. In preferred embodiments, the deimmunized anti-CR1 antibody comprises one or more non-human V_{H} or V_{L} sequences modified to comprise one or more amino acid substitutions so that the deimmunized antibody is non-immunogenic or less immunogenic to a human when compared to the respective unmodified non-human sequences (see WO 00/34317, WO 98/52976, and U.S. Provisional Application No. 60/458,869 filed on Mar. 28, 2003, all of which are incorporated herein by reference in their entirety).

Completely human antibodies are particularly desirable for therapeutic treatment of human patients. In one embodiment, fully human antibodies can be made using techniques that are known in the art. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make antibodies are described in U.S. Pat. Nos. 6,150,584; 6,458,592; 6,420,140.

The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65 93). For a detailed discussion of this technology for producing human antibodies and human monoclonal antibodies and protocols for producing such antibodies, see e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.; see, for example, U.S. Pat. No. 5,985,615) and Medarex, Inc. (Princeton, N.J.), can be engaged to provide human antibodies directed against human CR1 using technology similar to that described above.

Completely human antibodies which recognize and bind a selected epitope can also be generated using a technique referred to as “guided selection.” In this approach a selected non human monoclonal antibody, e.g., a mouse antibody, is used to guide the selection of a completely human antibody recognizing the same epitope (Jespers et al., 1994, Bio/technology 12:899-903).

A pre-existing anti-CR1 antibody that does not bind CR2 (i.e., one that is known in the art), including but not limited to 7G9 (Reist et al. 1994. Eur. J. Immunol. 24:2018), YZ-1 (Changelian et al. 1985. J. Immunol. 134:1851), and E11 (AXXORA, LLC (San Diego, Calif.)), also including H4 and H9 deimmunized versions of E11 (Biovation, Ltd. (Aberdeen, UK)), can also be used.

b. Antigen Binding Portions of Antibodies

In one embodiment, the moiety which specifically binds CR1 consists of an antigen binding portion of an antibody. In one embodiment, the antigen-binding portion that binds to Protein A does not comprise an Fc domain. For example, the constructs of the invention can comprise CR1-binding fragments of such anti-CR1 antibodies. Such fragments may be recombinantly produced and engineered, synthesized, or produced by digesting an anti-CR1 antibody with a proteolytic enzyme.

In a preferred embodiment, the antigen-binding portion is an Fab, an Fab′, an (Fab′)2, or an Fv fragment of an immunoglobulin molecule. Such an Fab, Fab′ or Fv fragment can be obtained, e.g., from a full antibody by enzymatic processing. For example, pepsin digests an antibody below the disulfide linkages in the hinge region to produce an (Fab′)₂ fragment of the antibody which is a dimer of the Fab composed of a light chain joined to a VH-CH1 by a disulfide bond. The (Fab′)₂ fragments may be reduced under mild conditions to reduce the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antigen binding portion includes antigen binding portions of antibodies produced by the modification of whole antibodies or those synthesized de novo.

Alternatively, such a fragment can be obtained from a phage display library by affinity screening and subsequent recombinant expressing (see, e.g., Watkins et al., Vox Sanguinis 78:72-79; U.S. Patent Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552 554, each of which is incorporated herein by reference in its entirety).

Yet another alternative is to use a “single chain” Fv fragment. Single-chain Fv (scFv) fragments can be constructed in a variety of ways. Although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). For example, the C-terminus of V_{H} can be linked to the N-terminus of V_{L}. Typically, a linker (e.g., (GGGGS)₄) is placed between V_{H} and V_{L}. However, the order in which the chains can be linked can be reversed, and tags that facilitate detection or purification (e.g., Myc-, His-, or FLAG-tags) can be included (tags such as these can be appended to any anti-CR1 antibody or antibody fragment of the constructs of the invention; their use is not restricted to scFv). For a review of scFv see Pluckthun in The Pharmacology of Monoclonal Antibodies, vol. 113, 269-315 (Rosenburg and Moore eds., Springer-Verlag, New York 1994).

In another preferred embodiment, a single chain Fv (scFv) fragment can be obtained, e.g., from a library of phage-displayed antibody fragments by affinity screening and subsequent recombinant expression.

In still another embodiment, the antigen-binding portion of the construct molecule is a single-chain antibody (scAb). As used herein, a single-chain antibody (scAb) includes antibody fragments consisting of an scFv fused with a constant domain, e.g., the constant K domain, of an immunoglobulin molecule. In another embodiment, the antigen-binding portion of the construct molecule is a Fab, Fab′, (Fab′)₂, Fv, scFv, or scAb fragment fused with a linker peptide of a desired length comprising a chosen amino acid sequence. In preferred embodiment, the linker peptide consists of 1, 2, 5, 10, or 20 amino acids. Exemplary linker peptides are known in the art.

In alternative embodiments, the anti-CR1 antibodies used in the constructs of the present invention can be heavy chain dimers or light chain dimers. Still further, an anti-CR1 antibody light or heavy chain, or portions thereof, for example, a single domain anti-CR1 antibody (DAb), can be used.

Also included in the term antibody fragments are diabodies. The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., 1993 Proc. Natl. Acad. Sci. USA 90: 6444-8.

B. Moieties Comprising Antigens or Antigen Binding Molecules

In one embodiment, a construct of the instant invention comprise at least one antigen (e.g., one, two, three, four, or more antigens) derived from among a wide range of pathogenic agents, tumor cells or toxins, or pathogenic proteins) against which the generation of an immune response might be prophylactically and/or therapeutically beneficial. Exemplary pathogens of the invention from which antigenic moieties are derived include, e.g. viruses, bacteria, fungi, parasites, and epitopes derived therefrom.

In another embodiment, a construct of the instant invention comprise a moiety which binds to at lest one antigen (e.g., one, two, three, four, or more antigens) derived from among a wide range of pathogenic agents, tumor cells or toxins, or pathogenic proteins) against which the generation of an immune response might be prophylactically and/or therapeutically beneficial. Exemplary pathogens of the invention to which binding molecules may bind include, e.g. viruses, bacteria, fungi, and parasites.

1. Pathogenic Agents

a. Bacteria

Examples of bacteria (or epitopes thereof) to which binding moleucles may bind include: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacterfreundii, Salmonella enterica Typhimurium, Salmonella enterica Typhi, Salmonella enterica Paratyphi, Salmonella enterica Enteridtidis, Shigella dysenteriae, Shigellaflexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacterfetus, Campylobacterjejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, and Staphylococcus saccharolyticus. In a particular embodiment, a construct of the invention comprises a binding molecule which binds to Staphylococcal protein A.

b. Viruses

Examples of viruses (or epitopes thereof) which may be included in the claimed constructs and/or to which binding moleucles may bind include: influenza virus hemagglutinin (Genbank accession no. J02132; Air, 1981, Proc. Natl. Acad. Sci. USA 78:7639-7643; Newton et al., 1983, Virology 128:495-501), human respiratory syncytial virus G glycoprotein (Genbank accession no. Z33429; Garcia et al., 1994, J. Virol.; Collins et al., 1984, Proc. Natl. Acad. Sci. USA 81:7683), measles virus hemagglutinin (Genbank accession no. M81899; Rota et al., 1992, Virology 188:135-142), herpes simplex virus type 2 glycoprotein gB (Genbank accession no. M14923; Bzik et al., 1986, Virology 155:322-333), poliovirus I VP1 (Emini et al., 1983, Nature 304:699), envelope glycoproteins of HIV I (Putney et al., 1986, Science 234:1392-1395), hepatitis B surface antigen (Itoh et al., 1986, Nature 308:19; Neurath et al., 1986, Vaccine 4:34), diphtheria toxin (Audibert et al., 1981, Nature 289:543), streptococcus 24M epitope (Beachey, 1985, Adv. Exp. Med. Biol. 185:193), gonococcal pilin (Rothbard and Schoolnik, 1985, Adv. Exp. Med. Biol. 185:247), pseudorabies virus g50 (gpD), pseudorabies virus II (gpB), pseudorabies virus gIII (gpC), pseudorabies virus glycoprotein H, pseudorabies virus glycoprotein E, transmissible gastroenteritis glycoprotein 195, transmissible gastroenteritis matrix protein, swine rotavirus glycoprotein 38, swine parvovirus capsid protein, Serpulina hydodysenteriae protective antigen, bovine viral diarrhea glycoprotein 55, Newcastle disease virus hemagglutinin neuraminidase, swine flu hemagglutinin, swine flu neuraminidase, foot and mouth disease virus, hog colera virus, swine influenza virus, African swine fever virus, Mycoplasma hyopneumoniae, infectious bovine rhinotracheitis virus (e.g., infectious bovine rhinotracheitis virus glycoprotein E or glycoprotein G), or infectious laryngotracheitis virus (e.g. , infectious laryngotracheitis virus glycoprotein G or glycoprotein I), a glycoprotein of La Crosse virus (Gonzales Scarano et al., 1982, Virology 120 :42), neonatal calf diarrhea virus (Matsuno and Inouye, 1983, Infection and Immunity 39:155), Venezuelan equine encephalomyelitis virus (Mathews and Roehrig, 1982, J. Immunol. 129:2763), punta toro virus (Dalrymple et al., 1981, Replication of Negative Strand Viruses, Bishop and Compans (eds.), Elsevier, NY, p. 167), murine leukemia virus (Steeves et al., 1974, J. Virol. 14:187), mouse mammary tumor virus (Massey and Schochetman, 1981, Virology 115:20), hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651 693; Tiollais et al., 1985, Nature 317:489 495), of equine influenza virus or equine herpesvirus (e.g., equine influenza virus type A/Alaska 91 neuraminidase, equine influenza virus type A/Miami 63 neuraminidase, equine influenza virus type A/Kentucky 81 neuraminidase equine herpesvirus type 1 glycoprotein B, and equine herpesvirus type I glycoprotein D, antigen of bovine respiratory syncytial virus or bovine parainfluenza virus (e.g., bovine respiratory syncytial virus attachment protein (BRSV G), bovine respiratory syncytial virus fusion protein (BRSV F), bovine respiratory syncytial virus nucleocapsid protein (BRSV N), bovine parainfluenza virus type 3 fusion protein, and the bovine parainfluenza virus type 3 hemagglutinin neuraminidase), bovine viral diarrhea virus glycoprotein 48 or glycoprotein 53.

Other exemplary viruses include: hepatitis type A, hepatitis type B, hepatitis type C, influenza, varicella, adenovirus, herpes simplex type I (HSV I), herpes simplex type II (HSV II), rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV I), and human immunodeficiency virus type II (HIV II), any picornaviridae, enteroviruses, caliciviridae, any of the Norwalk group of viruses, togaviruses, such as alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein Barr virus, human herpesvirus 6, cercopithecine herpes virus I (B virus), and poxviruses.

c. Fungi

Examples of fungi (or epitopes thereof) which may be included in the claimed constructs and/or to which binding moleucles may bind include fungi from the genus Mucor, Candida, and Aspergillus, e.g., Mucor racmeosus, Candida albicans, and Aspergillus niger.

d. Parasites

Examples of parasites (or epitopes thereof) which may be included in the claimed constructs and/or to which binding moleucles may bind include: Toxoplasna gondii, Treponema pallidun, Malaria, and Cryptosporidium

e. Microbial Toxins

Examples of microbial toxins (or epitopes thereof) which may be included in the claimed constructs and/or to which binding molecules may bind include: e.g., toxins produced by Bacillus anthracis, Bacillus cereus, Bordatella pertussis, Clostridium botulinum, Clostridium perfringens, Clostridium tetani, Croynebacterium diptheriae, Salmonella sp. Shigella sp., Staphyloccus sp., and Vibrio cholerae.

Toxins such as ricin from jack bean and other naturally-occurring (e.g., produced by an organism) and man-made toxins or portions thereof may also be included in the subject constructs.

f. Vaccine Strains of Pathogens

In one embodiment, a construct of the invention comprises and/or binds to a vaccine strain of a pathogen.

A common vaccine strategy involves use of a live vaccine strain. Live vaccines include live attenuated pathogens, live recombinant vaccines, and heterologous vaccines. Live attenuated vaccines are viruses whose virulence has been reduced by in vitro culture manipulation (such as changed temperature or chemical modification). These live attenuated viruses replicate in the vaccine recipient without causing the standard disease pathology while still eliciting both cell mediated immunity and antibody response that subsequently recognizes the original virulent pathogen. Live recombinant vaccines are similar to live attenuated vaccines in that they originate from the virulent pathogen but are altered to decrease virulence by genomic alterations. Accordingly, live recombinant vaccines induce long-term humoral and cell mediated immune responses. Heterologous vaccines are pathogens closely related to the virulent pathogen of interest that share common antigens and replicate within the host without causing disease. Like live attenuated and live recombinant vaccines, heterologous vaccines induce a long-term humoral and cell mediated immune response.

A safer alternative to live vaccines are killed or inactivated vaccines. Killed and inactivated vaccines are either whole killed vaccines or subunit vaccines. Whole killed vaccines are made by culturing the pathogen in vitro and subsequently killing them (e.g., with beta-propiolactone or formaldehyde). After this treatment, the vaccine is unable to replicate and is therefore relatively safe.

Subunit vaccines are used when the known correlates of immunity suggest that immunity is raised against one or a few pathogen antigens. Subunit vaccines are made by culturing large amounts of the pathogen and then purifying for the proteins/antigens of interest. Recombinant subunit vaccines are immunogenic proteins of virulent organisms that are made by expressing the antigen's gene in an expression vector. Like inactivated vaccines, recombinant subunit vaccines only induce B cell antibody protection against the antigen. Because of the extreme genetic variability of certain pathogens (especially viruses), only highly conserved antigens can be considered for a recombinant subunit vaccine.

g. Pathogenic Proteins

The antigenic second moiety of certain constructs of the invention may comprise a pathogenic protein and/or a molecule which binds to a pathogenic protein, e.g., one or more endogenous or exongenous proteins that are associated with a disease state or condition, such as a particular cancer, bacterial or viral infection. Specific examples of such pathogenic proteins include, e.g., amyloid protein, prion proteins, PSA, etc., and the second moiety of constructs may comprise antigenic epitopes/polypeptides derived therefrom.

h. Antibodies Against Which Anti-Idiotypic Antibodies are Desired

The antigenic second moiety of certain constructs of the invention may comprise portions of antibodies that provoke an anti-idiotypic antibody response in a host. Anti-idiotypes are monoclonal antibodies directed to the antigen recognition site of other antibodies. Anti-idiotypes can regulate the immune system and other biological processes. Methods of making anti-idiotypic antibodies are known in the art. (See, e.g., Antibodies, Antigens. and Molecular Mimicry, Volume 178: Antibodies, Antigens and Molecular Mimicry Editor-in-chief Abelson, John N. Editor-in-chief Simon, Melvin I. Volume editor (United Kingdom) and references cited therein.) Anti-idiotypic antibodies are known to be useful in a variety of settings, including treatment of cancer (see, e.g., Wettendorff, M., et al. . 1990. Modulation of anti-tumor immunity by anti-idiotypic antibodies. In: Idiotypic Network and Diseases (J. Cerny and J. Hiernaux, eds.). Am. Soc. Microbiol., Washington, DC, pp 203-229). In addition, anti-idiotypic antibodies that possess the internal image of antigen can induce protective humoral immunity toward microbes. For example, antigen mimicry by monoclonal anti-idiotypes of a distinct epitope of the human immunodeficiency virus (HIV) envelope protein that is defined by a synthetic peptide induced antibodies in three mammalian species that interacted with HIV-1 gp120 and inhibited in vitro syncytium formation caused by HIV-1, IIIB and MN isolates. (Zaghouani et al. 1991. PNAS. 88: 5645-5649).

i. Epitopes

In one embodiment, a construct of the invention comprises an epitope of an antigen. Epitopes may be derived from and/or comprise protein, (poly)peptide, carbohydrate, lipid, lipopolysaccharide, polysaccharide, small molecule(s), peptidoglycan and/or glycoprotein.

Epitopes appropriate for inclusion in the subject constructs can be prepared using standard methods. The epitope of an antigen used in the subject constructs may be of varying length, although it is generally preferred that the portions be at least 9 amino acids long. It will be understood that more than one epitope can be included in a construct of the invention. In one embodiment, a construct of the invention comprises the entire molecule to which an immune response is desired, e.g., a complete pathogen.

In selecting epitopes, the major consideration for B cell epitopes is accessibility on the surface of the pathogen and the preservation of protein conformation in developing epitopes for accurate antibody recognition of the antigen.

T lymphocytes are specific for peptides presented in the context of HLA molecules (human MHC—histocompatibility complex molecules). Peptides are processed in the cytosol of Antigen Presenting Cells (APCs) via limited proteolytic fragmentation of available proteins, transported to the endoplasmic reticulum where they are bound to HLA molecules. The HLA-peptide complex is then exported to the cell's surface and presented to T cells, e.g., CTLs. An important factor in this process is the specificity of the HLA molecules for the different peptides. HLA molecules are extremely polymorphic and vary from person to person and race to race. Accordingly, T cell vaccine development is often restricted by HLA types. Therefore, selection of T cell epitopes is primarily governed by epitope conservation, proteosome processing, and HLA selectivity. However, almost all HLA types can be categorized by nine “HLA supertypes”—each supertype selective for sequentially similar peptides . See, e.g., March, S., et al., HLA Facts Book, Academic Press, 2000.

Molecular sequence data for many pathogens are available in many public databases. Such sequence data can be employed in either overlapping epitope approaches or bioinformatics approaches to identify T cell epitopes. (See, e.g., Brusic et al. 2005. Expert Rev. Vaccines 4:407 and references cited therein).

In one embodiment, an overlapping approach is used to identify T cell epitopes. For example, partially overlapping peptides (e.g., 10 aa long peptides overlapping by 5 amino acids) covering the entire amino acid sequence of the protein of interest are made and then screened for their ability to bind HLA molecules or to induce a T cell response.

In another embodiment, bioinformatics prediction methods can be used for identification of HLA-binding peptides, including, binding motifs, quantitative matrices, decision trees, artificial neural networks, hidden Markov models, and molecular modeling. Novel T-cell epitopes have been discovered using computation predictions for antigens such as cancer antigens (Dong et al. 2004. Cancer Biol. Ther. 3:891; Consogno et al. 2003 Blood 101:1038), autoantigens (Flynn et al. 2004. Cell. Immunol. 229:79), pathogen antigens (DeGroot et al. 2003. Vaccine 21:4486; Al-Attiyah and Mustafa. 2004. Scand. J. Immunol. 59:16; Brusic et al. 2001. J. Mol. Graph. Model. 19:405), and allergens (DeLalla et al. 1999. J. Immunol. 163:1725). Exemplary algorithms which can be used in epitope evaluation include: the EpiMatrix algorithm, the ClustiMer algorithm, and the Conservatrix algorithm. (See, e.g., Sbai, et al. 2001. In one embodiment, T cell epitopes may be predicted utilizing a computer algorithm such as TSITES (MedImmune, Md.), in order to scan for potential T-helper sites and CTL sites. Current Drug Targets—Infectious Disorders 1:303 and references cited therein).

For example, the process may begin with identification of a target protein thought to play a role in a disease process. A library of overlapping amino acid sequences spanning the entire length of the protein is then synthesized. A binding assay is performed for each of the test peptides by introducing a buffer designed to unfold and disassociate the MHC and placeholder peptide in a microtitre well. The placeholder peptide and beta 2 microglobulin are washed away, leaving the unfolded MHC bound to the reaction well. A peptide from the synthesized library and additional beta 2 microglobulin are added to each well and incubated in a buffer designed to promote refolding of the complex.

A fluorescent-labeled antibody designed to recognize only a properly folded peptide/MHC complex is added to each well. This step provides the identification of those test peptides which bind to the MHC and warrant additional analysis to characterize their binding affinity and rate of dissociation. Peptides that do not bind to the MHC are clearly identified and eliminated from further study.

Successful peptide-FLA complexes (e.g., for the nine identified HLA supertypes) could then be delivered via several vaccine methods. One vaccine approach is integration of all nine peptide-BLA-supertype complexes into a polytope vaccine design. (Thomson S A, et al. Proc Natl Acad Sci USA 92: 5845-5849). Polytope, or polyepitope vaccines have been shown to successfully elicit immune responses to large numbers of epitopes expressed in a single viral or DNA vector.

From this analysis, peptides can be synthesized and used as targets in an in vitro cytotoxic assay. Other assays may also be utilized, including, for example, ELISA, or ELISPOT, which detects the presence of antibodies against the newly introduced vector, as well as assays which test for T helper cells, such as gamma-interferon assays, IL-2 production assays and proliferation assays.

Epitopes which are immunogenic may be selected by other art recognized methods. For example, the HLA A2.1 transgenic mouse has been shown to be useful as a model for human T-cell recognition of viral antigens. Briefly, in the influenza and hepatitis B viral systems, the murine T cell receptor repertoire recognizes the same antigenic determinants recognized by human T cells. In both systems, the CTL response generated in the HLA A2.1 transgenic mouse is directed toward virtually the same epitope as those recognized by human CTLs of the HLA A2.1 haplotype (Vitiello et al. (1991) J. Exp. Med. 173:1007-1015; Vitiello et al. (1992) Abstract of Molecular Biology of Hepatitis B Virus Symposia).

In another embodiment, a portion of antigen may be obtained by truncating the coding sequence at various locations including, for example, to include one or more domains of a pathogen's genome. For example, for an HIV pathogen, such domains include structural domains such as Gag, Gag-polymerase, Gag-protease, reverse transcriptase (RT), integrase (IN) and Env. The structural domains are often further subdivided into polypeptides, for example, p55, p24, p6 (Gag); p160, p10, p15, p31, p65 (pol, prot, RT and IN); and gp160, gp120 and gp41 (Ems) or Ogp140 as constructed by Chiron Corporation. Molecular variants of such polypeptides may also be used, for example as described in PCT/US99/31245; PCT/US99/31273 and PCT/US99/31272.

The epitopes of this invention can be optimized (increased in immunogenicity) using methods known in the art, e.g., so that they induce a higher immune response. For example, polynucleotide sequences that encode certain pathogen-derived antigens (e.g., Ogp140 of HIV) can be optimized by codon substitution of wild type sequences.

In another embodiment, an epitope (or a construct) may be directly modified to enhance immunogenicity or physical properties such as stability. For example, cyclization or circularization of a peptide can increase the peptide's antigenic and immunogenic potency. See, e.g., U.S. Pat. No. 5,001,049 which is incorporated by reference herein.

The immunogenicity of certain epitopes (or constructs) may also be modulated by coupling to fatty acid moieties to produce lipidated peptides. Convenient fatty acid moieties include glycolipid analogs, N-palmityl-S-(2RS)-2,3-bis-(palmitoyloxy)propyl-cysteinyl-serine (PAM3 Cys-Ser), N-palmityl-S-[2,3 bis (palmitoyloxy)-(2RS)-propyl-[R]-cysteine (TPC), tripalmitoyl-S-glycerylcysteinlyseryl-serine (P_{3CSS}), or adipalmityl-lysine moiety.

An epitope or construct of the invention may also be conjugated to a lipidated amino acid, such as an octadecyl ester of an aromatic acid, such as tyrosine, including actadecyl-tryrosine (OTH).

j. Tumor Cell Antigens

Other suitable antigens to which binding moleucles may bind include tumor-associated antigens for the prevention or treatment of cancers. Examples of tumor-associated antigens include, but are not limited to, phCG, gp100 or Pmell7, HER2/neu, CEA, gp100, MART1, TRP-2, melan-A, NY-ESO-1, MN (gp250), idiotype, MAGE-1, MAGE-3, Tyrosinase, Telomerase, MUC-1 antigens, and germ cell derived tumor; antigens. Tumor associated antigens also include the blood group antigens, for example, Lea, Leb, LeX, LeY, H-2, B-1, B-2 antigens. Alternatively, more than one antigen can be included within the constructs of the invention. For example, a MAGE antigen can be combined with other antigens such as melanin A, tyrosinase, and gp100 along with adjuvants such as GM-CSF or IL-12, and linked to an anti-CR1-specific antibody.

For example, CD20 is a pan B antigen that is found on the surface of both malignant and non-malignant B cells that has proved to be an extremely effective target for immunotherapeutic antibodies for the treatment of non-Hodgkin's lymphoma. In this respect, pan T cell antigens such as CD2, CD3, CD5, CD6 and CD7 also comprise tumor associated antigens within the meaning of the present invention. Still other exemplary tumor associated antigens comprise but not limited to MAGE-1, MAGE-3, MUC-1, HPV 16, HPV E6 & E7, TAG-72, CEA, L6-Antigen, CD19, CD22, CD37, CD52, HLA-DR, EGF Receptor and HER2 Receptor. In many cases immunoreative antibodies for each of these antigens have been reported in the literature.

C. Molecules Which Bind to Antigen

In one embodiment, the second moiety of a construct may comprise one or more molecules (e.g., one, two, three, four, or more molecules) that bind an antigen. Such molecules are art-recognized, and include, e.g., antibodies which bind to antigens or antigen binding portions of such antibodies. In one embodiment, the construct in addition to comprising a moiety that binds to antigen may further comprise the antigen to which the moiety binds. Examples of antigen binding moieties are described below.

1. Antibodies

In one embodiment, the antigen-binding moiety of a construct of the invention is a monoclonal antibody which binds to an antigen or epitope derived therefrom. Such an antibody may recognize a pathogen, e.g., such as is known in the art an exemplary pathogen set forth herein. Methods for producing monoclonal antibodies are known in the art (see, e.g., Kohler and Milstein, Nature 256:495-497, 1975 and Hurrell, J. G. R., Ed., Monoclonal Hybridoma Antibodies: Techniques and Applications, CRC Press, Inc., Boca Raton, Fla., 1982, which are incorporated herein by reference), as well as techniques for stably introducing immunoglobulin-encoding DNA into myeloma cells (see, e.g., Oi et†al., Proc. Natl. Acad. Sci. USA 80:825-829, 1983; Neuberger, EMBO J. 2:1373-1378, 1983; and Ochi et al., Proc. Natl. Acad. Sci. USA 80:6351-6355, 1983). These techniques, which include in vitro mutagenesis and DNA transfection, allow for the construction of recombinant immunoglobulins; these techniques can be used to produce the antigen-binding antibodies used in certain constructs of the invention. Alternatively, the antigen-binding antibodies can be obtained from a commercial supplier.

Alternatively, the antigen-binding moiety of certain constructs of the invention can be a polyclonal antibody. Methods for preparing polyclonal antibodies are well known in the art (see, for example, Sambrook et al., Molecular Cloning: A Laboratory Manual, Second Edition, Cold Spring Harbor, N.Y., 1989, which is incorporated herein by reference). As would be evident to one of ordinary skill in the art, polyclonal antibodies can be generated from a variety of warm-blooded animals, such as horses, cows, goats, sheep, dogs, chickens, rabbits, mice, and rats. The antigen-binding antibody of certain constructs of the invention can be an antibody of the IgA, IgD, IgE, IgG, or IgM class.

In one embodiment, the moiety comprising an antigen binding portion which binds to Staphylococci may be any antibody or antigen binding portion thereof that recognizes a Staphylococcal surface antigen, e.g., protein A. Exemplary antibodies are commercially available. In one embodiment, the anti-protein A antibody is SPA 27 (Catalog # P 2921 from Sigma Aldrich (St, Louis Mo.)). Although in the description below the protein A antigen is often referred to for simplicity, it will be understood that antibodies recognizing other Staphylococcal surface antigens or antigen binding portions thereof can also be incorporated into the subject bispecific molecules. Other exemplary molecules for targeting a S. aureus surface proteins that hinder bacterial attachment to cells (e.g., Staphylococcal matrix binding proteins or adhesions).

In one embodiment, the antigen-binding moiety of a construct of the invention is a monoclonal antibody which binds to an antigen or epitope derived therefrom. Such an antibody may recognize a pathogen, e.g., such as is known in the art an exemplary pathogen set forth herein. Methods for producing monoclonal antibodies are known in the art (see, e.g., Kohler and Milstein, Nature 256:495-497, 1975 and Hurrell, J. G. R., Ed., Monoclonal Hybridoma Antibodies: Techniques and Applications, CRC Press, Inc., Boca Raton, Fla., 1982, which are incorporated herein by reference), as well as techniques for stably introducing immunoglobulin-encoding DNA into myeloma cells (see, e.g., Oi et†al., Proc. Natl. Acad. Sci. USA 80:825-829, 1983; Neuberger, EMBO J. 2:1373-1378, 1983; and Ochi et al., Proc. Natl. Acad. Sci. USA 80:6351-6355, 1983). These techniques, which include in vitro mutagenesis and DNA transfection, allow for the construction of recombinant immunoglobulins; these techniques can be used to produce the antigen-binding antibodies used in certain constructs of the invention. Alternatively, the antigen-binding antibodies can be obtained from a commercial supplier.

Alternatively, the antigen-binding moiety of certain constructs of the invention can be a polyclonal antibody. Methods for preparing polyclonal antibodies are well known in the art (see, for example, Sambrook et al., Molecular Cloning: A Laboratory Manual, Second Edition, Cold Spring Harbor, N.Y., 1989, which is incorporated herein by reference). As would be evident to one of ordinary skill in the art, polyclonal antibodies can be generated from a variety of warm-blooded animals, such as horses, cows, goats, sheep, dogs, chickens, rabbits, mice, and rats. The antigen-binding antibody of certain constructs of the invention can be an antibody of the IgA, IgD, IgE, IgG, or IgM class.

In one embodiment, the moiety which binds an antigen consists of an antigen binding portion of an antibody. In one embodiment, the antigen-binding portion that binds to Protein A does not comprise an Fc domain. Such fragments may be recombinantly produced and engineered, synthesized, or produced by digesting an antibody with a proteolytic enzyme.

In a preferred embodiment, the antigen-binding portion is an Fab, an Fab′, an (Fab′)2, or an Fv fragment of an immunoglobulin molecule. Such an Fab, Fab′ or Fv fragment can be obtained, e.g., from a full antibody by enzymatic processing. For example, pepsin digests an antibody below the disulfide linkages in the hinge region to produce an (Fab′)₂ fragment of the antibody which is a dimer of the Fab composed of a light chain joined to a VH-CH1 by a disulfide bond. The (Fab′)₂ fragments may be reduced under mild conditions to reduce the disulfide linkage in the hinge region thereby converting the (Fab′)₂ dimer to a Fab′ monomer. The Fab′ monomer is essentially an Fab with part of the hinge region. See Paul, ed., 1993, Fundamental Immunology, Third Edition (New York: Raven Press), for a detailed description of epitopes, antibodies and antibody fragments. One of skill in the art will recognize that such Fab′ fragments may be synthesized de novo either chemically or using recombinant DNA technology. Thus, as used herein, the term antigen binding portion includes antigen binding portions of antibodies produced by the modification of whole antibodies or those synthesized de novo.

Alternatively, such a fragment can be obtained from a phage display library by affinity screening and subsequent recombinant expressing (see, e.g., Watkins et al., Vox Sanguinis 78:72-79; U.S. Pat. Nos. 5,223,409 and 5,514,548; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; and McCafferty et al., 1990, Nature 348:552 554, each of which is incorporated herein by reference in its entirety).

Yet another alternative is to use a “single chain” Fv fragment. Single-chain Fv (scFv) fragments can be constructed in a variety of ways. Although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). For example, the C-terminus of V_{H} can be linked to the N-terminus of V_{L}. Typically, a linker (e.g., (GGGGS)₄) is placed between V_{H} and V_{L}. However, the order in which the chains can be linked can be reversed, and tags that facilitate detection or purification (e.g., Myc-, His-, or FLAG-tags) can be included (tags such as these can be appended to any anti-CR1 antibody or antibody fragment of the constructs of the invention; their use is not restricted to scFv). For a review of scFv see Pluckthun in The Pharmacology of Monoclonal Antibodies, vol. 113, 269-315 (Rosenburg and Moore eds., Springer-Verlag, New York 1994).

In another preferred embodiment, a single chain Fv (scFv) fragment can be obtained, e.g., from a library of phage-displayed antibody fragments by affinity screening and subsequent recombinant expression.

In still another embodiment, the antigen-binding portion of the construct molecule is a single-chain antibody (scAb). As used herein, a single-chain antibody (scAb) includes antibody fragments consisting of an scFv fused with a constant domain, e.g., the constant K domain, of an immunoglobulin molecule. In another embodiment, the antigen-binding portion of the construct molecule is a Fab, Fab′, (Fab′)₂, Fv, scFv, or scAb fragment fused with a linker peptide of a desired length comprising a chosen amino acid sequence. In preferred embodiment, the linker peptide consists of 1, 2, 5, 10, or 20 amino acids.

In alternative embodiments, the antibody used in the constructs of the present invention can be heavy chain dimers or light chain dimers. Still further, an antibody light or heavy chain, or portions thereof, for example, a single domain antibody (DAb), can be used.

Also included in antibody fragments are diabodies. The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., 1993 Proc. Nat]. Acad. Sci. USA 90: 6444-8.

It will be understood that antibodies made or altered to have reduced immunogenicity in humans (e.g., chimeric antibodies, humanized antibodies, deimmunized antibodies, or fully human antibodies) can be included in the subject constructs. Techniques for making such antibodies are known in the art and exemplary methods are described herein above for CR1 antibodies.

2. Soluble Forms of Cellular Receptors

In one embodiment of the invention, a moiety that binds an antigen is an agent that has a cellular receptor. For example, certain viruses have specific receptors on cells which are responsible for viral tropism and uptake. Soluble forms of such receptors can be made and incorporated into the subject constructs.

Well-characterized examples of cell surface receptors that may be used in the constructs of the present invention include, e.g., CD4 and CCR5 for HIV, ICAM-1 for many human rhinoviruses, PVR (poliovirus receptor) for poliovirus, aminopeptidase N for many human coronaviruses, cell surface proteoglycans containing heparan sulfate moieties and possibly FGF for HSV, and CR2 for EBV, with the preceding list of receptors among the most extensively characterized (refer to Norkin 1995 Clin. Microbiol. Reviews 8: 293-315). In general, production of soluble forms of such cell surface receptor molecules may be performed as for any membrane-spanning and/or membrane-tethered protein, via a process involving truncation of the protein and/or nucleotide sequence encoding the protein (and/or the removal of a membrane anchor), resulting in deletion of the transmembrane and/or membrane-anchoring domain(s) of the protein. Such production of soluble forms of cell surface receptors may involve methods as described in, e.g., Greve, J M et al. 1991 J. Virol. 65: 6015-6023 and Marlin SD et al. 1990 Nature 344: 70-72 (production of soluble forms of ICAM-1), and Schooley R T et al. 1990 Ann. Intern. Med. 112: 247-253, Daar ES et al, 1990 Proc. Natl. Acad. Sci. USA 87:6574-6578, Kahn J O et al. 1990 Ann. Intern. Med. 112: 254-261, and O'Brien W A et al. 1990 Nature 348: 69-73 (production of recombinant soluble forms of CD4). The contents of each of these cited documents are incorporated in their entirety herein by reference. One of skill in the art will also recognize that soluble forms of any cell surface receptor may be made via use of such methods.

3. Antigen Binding Small Molecules or Drugs

In one embodiment, a small molecule or drug that specifically binds to an antigen can be incorporated into a construct of the invention. For example, amphotericin B is known to preferentially bind to the primary fungal cell membrane sterol, ergosterol. This binding disrupts osmotic integrity of the fungal membrane, resulting in leakage of intracellular potassium, magnesium, sugars, and metabolites and then cellular death (refer to Terrell, C L, and Hughes, C E, 1992 Mayo Clin Proc. 67:69-91, incorporated herein by reference). In one embodiment, a construct of the invention comprises amphotericin B as a second moiety. One of skill in the art will readily recognize that additional small molecules or drugs that specifically bind to an antigen may also be used in the constructs of the present invention.

### III. METHODS OF MAKING CONSRUCTS OF THE INVENTION

The present invention provides a construct comprising a first moiety comprising a CR1-binding portion that does not bind CR2 and a second moiety comprising an antigen or a molecule that binds to the antigen

In the present invention, the CR1-specific binding moiety and the antigen to which an immune response is desired or a molecule that binds thereto can be linked using methods known in the art, e.g., covalently or non-covalently. Exemplary linking methodology includes but is not limited to, chemical cross-linking. In yet another embodiment, the construct molecule is produced by an art recognized method other than chemical cross-linking, including but not limited to, methods involving fusion of hybridoma cell lines, recombinant techniques, and protein trans-splicing. See e.g., PCT publication WO 02/46208 and PCT publication WO 01/80883, all of which are incorporated herein by reference in their entirety. Exemplary means of linking the first and second moieties of the subject constructs are described in further detail below.

A. Crosslinking

In specific embodiments of the invention, the construct comprises an anti-CR1 mAb cross-linked to one or more antigens to which an immune response is desired or to one or more antigen-binding molecules that bind such antigen(s). In specific embodiments, the construct comprises an anti-CR1 mAb cross-linked to at least 1, 2, 3, 4, 5 or 6 antigens or antigen-binding molecules. Preferably, the CR1-specific moieties are attached to the antigen such that CR1 binding is not compromised. In preferred embodiments, a construct of the invention binds CR1 with an activity (e.g., affinity or avidity) at least 5%, 15%, 25%, 50%, 90% or 99% of that of the native CR1-binding moiety (e.g., native antibody) from which the CR1-binding portion is derived.

In one embodiment, the antigen to which an immune response is desired or the antigen binding molecule is attached at a predetermined site to the moiety that binds CR1. Preferably, such a predetermined site is selected so that the CR1-binding affinity of the CR1-specific binding moiety and the antigenicity of the antigen to which an enhanced immune response is provoked are not comprised. More preferably, such a predetermined site is a site on the surface (i.e., a site which is exposed) of the CR1-specific binding moiety. In a preferred embodiment, the or antigen binding molecule is attached to the CR1-specific binding moiety via a cysteine residue in the antigen and/or CR1-binding moiety.

If more than one antigen or antigen binding molecule is cross-linked to one moiety that binds to CR1, the molecules can be the same (e.g., derived from the same pathogenic peptide and/or the same epitope) or different (e.g., derived from distinct pathogenic peptides) or can bind the same or different antigens.

In one embodiment, the two moieties of a construct are preferably conjugated by cross-linking via a cross-linker (cross-linking agent). Exemplary cross-linking chemistries are known in art. In a preferred embodiment of the invention, the CR1 binding moiety and the antigen or antigen binding moiety are linked using cross-linking agents sulfosuccinimidyl 4 (N maleimidomethyl) cyclohexane 1 carboxylate (sSMCC) or N-succinimidyl-S-acetyl thioacetate (SATA). In another embodiment of the invention, the CR1-binding moiety and the moiety comprising an antigen or antigen binding molecule are conjugated via a poly-(ethylene glycol) cross-linker (PEG). In this embodiment, the PEG moiety can have any desired length. For example, the PEG moiety can have a molecular weight in the range of 200 to 20,000 Daltons. Preferably, the PEG moiety has a molecular weight in the range of 500 to 1000 Daltons or in the range of 1000 to 8000 Daltons, more preferably in the range of 3250 to 5000 Daltons, and most preferably about 5000 Daltons. Such a construct can be produced using cross-linking agents N-succinimidyl-S-acetyl thioacetate (SATA) and a poly(ethylene glycol)-maleimide, e.g., monomethoxy poly(ethylene glycol)-maleimide (mPEG-MAL) or NHS-poly(ethylene glycol)-maleimide (PEG-MAL). Methods of producing, e.g., PEG-linked bispecific molecules is described in U.S. Provisional Application No. 60/411,731, filed on Sep. 16, 2002, which is incorporated herein by reference.

In still another embodiment, the antigen to which an enhanced immune response is induced, or the antigen-binding molecule that binds such an antigen, is produced with a free thiol by an appropriate host cell (see, e.g., Carter, U.S. Pat. No. 5,648,237, which is incorporated herein by reference in its entirety), and the construct is produced by reacting the free thiol-containing antigen or antigen binding moiety with an appropriately derivatized, e.g., sSMCC derivatized, CR1 binding moiety. A moiety that binds CR1 with a free thiol can also be produced directly, i.e., without using a chemical cross-linker, e.g., a maleimide. For example, in another embodiment, the construct comprises a monoclonal anti-CR1 binding moiety (e.g., an antibody) conjugated with an antigen or antigen binding molecule via a disulfide bond. In one embodiment, such a construct can be produced by mixing an antigenic or antigen binding moiety having a free thiol with a CR1 binding moiety with a free thiol.

B. Genetic Fusion

In another embodiment, the construct comprises a moiety that binds to CR1 and an antigen and/or antigen-binding moiety linked by methods that do not involve chemical cross-linking. Fusion proteins of the invention are chimeric molecules which comprise a CR1-specific binding moiety and a second moiety comprising an antigen or antigen binding moiety. Fusion proteins can be made using methods known in the art. For example, the fusion proteins of the invention may be constructed as described in U.S. Pat. No. 6,194,177, PCT publication WO 02/46208; and PCT publication WO 01/80883). Additionally, the subject fusion proteins can be made employing methods used to make chimeric antibodies in which a variable domain from an antibody of one species is substituted for the variable domain of another species. See, for example, EP 0 125 023; Munro, Nature 312: (13 Dec. 1984); Neuberger et al., Nature 312: (13 Dec. 1984); Sharon et al., Nature 309: (24 May 1984); Morrison et al., Proc. Natl. Acad. Sci. USA 81:6851-6855 (1984); Morrison et al., Science 229:1202-1207 (1985); and Boulianne et al., Nature 312:643-646 (13 Dec. 1984). In general, the DNA encoding the first moiety of a construct is cloned by PCR and ligated, in frame, into DNA encoding second moiety of the construct. DNA encoding the fusion protein is transfected into a host cell for expression. The sequence of the final construct can be confirmed by sequencing. In one embodiment, when preparing the fusion proteins of the present invention, a nucleic acid molecule encoding the first moiety will be fused in frame C-terminally to nucleic acid molecule encoding the N terminus of the second moiety. N-terminal fusions are also possible in which the second moiety is fused to the N-terminus of the first moiety. The precise site at which the fusion is made is not critical; particular sites may be selected in order to optimize the biological activity, secretion, or binding characteristics of the molecule. Other methods of making fusion proteins are taught, e.g., in WO0069913A1, WO0040615A2, U.S. Pat. Nos. 5,116,964 and 5,225,538.

In addition, PCT publication WO 01/80883 describes bispecific molecules produced by methods involving fusion of hybridoma cell lines, recombinant techniques, and in vitro reconstitution of heavy and light chains obtained from appropriate monoclonal antibodies. PCT publication WO 02/46208 describes bispecific molecules produced by protein trans-splicing.

C. Receptor Ligand Interaction

Receptors are molecules capable of specifically binding to a ligand by affinity-based interactions that do not involve complementary base pairing. A ligand and its corresponding receptor (e.g., EGF and EGFR, estrogen and the estrogen receptor, yeast alpha factor and the alpha factor receptor, etc.) form a specific binding pair. The strength of such receptor-ligand interactions may be exploited in certain constructs of the methods and compositions of the present invention, as such receptor-ligand pairings are capable of creating the linkage between the first and second moieties of the constructs of the invention. It will be readily apparent to one of skill in the art that any sufficiently robust receptor-ligand interaction can be used in certain constructs of the invention to create such a linkage.

### IV. PURIFICATION AND TESTING OF CONSTRUCTS

In one embodiment, the constructs produced by a method such as described supra are purified. Constructs can be purified by any method known to one skilled in the art using, e.g., molecular size or specific binding affinity or a combination thereof. In one embodiment, the constructs can be purified by ion exchange chromatography using columns suitable for isolation of the constructs of the invention including DEAE, Hydroxylapatite, Calcium Phosphate (see generally Current Protocols in Immunology, 1994, John Wiley & Sons, Inc., New York, N.Y.). In another embodiment, the constructs can be purified by size exclusion chromatography.

In another embodiment, constructs comprising a protein A binding region are purified by three-step successive affinity chromatography (Corvalan and Smith, 1987, Cancer Immunol. Immunother., 24:127-132): the first column is made of protein A bound to a solid matrix, wherein the Fc portion of an antibody present in the construct binds protein A, and wherein the antibodies bind the column; followed by a second column that utilizes the molecule to which a moiety of a construct of the invention binds (e.g., CR1 or an antigen) bound to a solid matrix; and followed by a third column that utilizes specific binding of the moiety to which an elevated immune response occurs, e.g., a column that presents an antibody to the antigen to which the immune response is enhanced. In another embodiment, any one of the above mentioned steps can be used individually.

The constructs can also be purified by a combination of size exclusion IPLC and affinity chromatography. In one embodiment, the appropriate fraction eluted from size exclusion HPLC is further purified using a column containing a molecule specific to the antigen of the construct, e.g., an antibody that can bind the antigen of the construct or other moiety known to interact with the construct antigen.

In another embodiment, a DNA sequence encoding an antibody or antigen binding portion thereof, or an antigen for induction of an immune response, is fused with the DNA sequence of a short peptide tag and introduced into cells to express a “tagged protein.” Since antibodies to the peptide tag are commercially available, such antibodies can be used to immunoaffinity purify the protein. Exemplary tags include, e.g., FLAG™, HA, HIS, c-Myc, VSV-G, V5 and HSV.

The activity of a construct, e.g., whether it can induce and/or enhance an immune response to an antigen in a subject, can be tested by an appropriate method known in the art.

Various constructs of the invention can be combined into a “cocktail” of constructs. Such cocktail of constructs can include, e.g., construct molecules each having a CR1 binding portion conjugated to at least one copy of an antigen or antigen binding molecule. For example, the construct cocktail comprises a plurality of different construct molecules, wherein each different construct molecule in the plurality contains a different antigen-binding moiety and/or antigen to which an immune response is desired. Such construct cocktails are useful as personalized medicine tailored according to the need of individual patients. Alternatively, a cocktail of constructs can include constructs each having a different CR1 binding moiety, e.g., a different antibody which binds a different site on CR1, conjugated to one or more antigen or antigen binding moieties.

### VI. CHARACTERIZATION OF CONSTRUCTS

The constructs of the invention can be characterized by various methods known in the art. The yield of constructs can be characterized based on the protein concentration. In one embodiment, the protein concentration is determined using a Lowry assay. Preferably, the construct produced by the method of the present invention has a protein concentration of at least 0.100 mg/ml, more preferably at least 2.0 mg/ml, still more preferably at least 5.0 mg/ml, most preferably at least 10.0 mg/ml. In another embodiment, the concentration of the constructs is determined by measuring UV absorbance. The concentration is determined as the absorbance at 280 nm. Preferably, the construct produced by the method of the present invention has an absorbance at 280 nm of at least 0.14.

A construct of the invention can also be characterized using other standard methods known in the art. For example, in one embodiment, high performance size exclusion chromatography (HPLC-SEC) assay is used to determine the content of contamination by, e.g., free IgG proteins. In preferred embodiments, the constructs produced by a method of the present invention have a contaminated IgG concentration of less than 6.0 mg/ml, more preferably less than 2.0 mg/ml, still more preferably less than 0.5 mg/ml, most preferably less than 0.03 mg/ml. In one embodiment, the constructs can be characterized by using SDS-PAGE to determine the molecular weight of the construct.

A construct can also be characterized based on the functional activity of the moieties comprising the construct, e.g., the effectiveness of the construct in enhancing an immune response to the antigen to which an enhanced immune response is targeted, can be tested using an in vivo or in vitro model.

For example, in one embodiment, an animal is exposed, e.g., to a microorganism and is treated with a construct comprising a CR1 binding molecule linked to an antigen or antigen binding molecule. One or more parameters of induction and/or enhancement of an immune response, such as antibody production, T cell activation, survival, symptoms, or microbial count (a count of colonies or infectious particles) from the animal can be assessed and compared with that observed in a control animal, an animal not treated with the construct.

In one embodiment, the ability to bind to CR1 is determined using ELISA with immobilized CR1 receptor molecules (attached to a solid phase, e.g., a microtiter plate) (see Porter et al., U.S. provisional application No. 60/380,211, which is incorporated herein by reference in its entirety). In a preferred embodiment, ELISA plates are prepared by incubating ELISA plates, e.g., high binding flat bottom ELISA plates (Costar EIA/RIA strip plate 2592) with a suitable amount of a bicarbonate solution of receptors. Preferably, the concentration of the bicarbonate solution of receptors is 0.2 ug/ml prepared from 5 mg/ml sCR1 receptors stock (Avant Technology Inc.) and a carbonate-bicarbonate buffer (pH 9.6, Sigma C-3041). In a preferred embodiment, 100 ul receptor-bicarbonate solution is dispensed into each well of the ELISA plates and the plates are incubated at 4° C. overnight. The plates are then preferably washed using, e.g., a wash buffer (PBS, 0.1% Tween-20, 0.05% 2-Chloroacetamide). In another preferred embodiment, a SuperBlock Blocking Buffer in PBS (Pierce) is added to the plates for about 30-60 min at room temperature after the wash. The plates can then be dried and stored at 4° C. The titration of anti-CR1-specific antibodies or constructs that bind CR1 but not CR2 can be carried out using, e.g., human anti-CR1 IgG, as the calibrator. In a preferred embodiment, the calibrator human anti-CR1 IgG has a concentration of 300 or 600 mg/ml. In one embodiment, the titration of the purified composition of a construct of the invention is carried out using PBS, 0.25% BSA, 0.1% Tween-20 as the diluent buffer, PBS, 0.1% Tween-20, 0.05% 2-Chloroacetamide as the wash buffer, TMB-Liquid Substrate System for ELISA (3,3′, 5,5′-Tetramethyl-Benzidine) and 2N H₂SO₄ as the stop solution. Preferably, the construct composition produced by the method of the present invention has a titer in such an assay of at least 0.10 mg/ml, more preferably at least 0.20 mg/ml, still more preferably at least 0.30 mg/ml, and most preferably at least 0.50 mg/ml. In some embodiments, a specific activity is determined. The specific anti-CR1 antibody activity is a ratio of titer and protein concentration as determined by Lowry or any other protein assay.

The antigen-binding activity can be determined using ELISA, e.g., using with immobilized antigen molecules.

### VII. USES OF CONSTRUCTS OF THE INVENTION

The constructs of the present invention are useful in treating or preventing a disease or disorder or other undesirable condition associated with the presence of a pathogenic and/or disease-associated antigenic molecule, neoplastic growth, or toxin.

The preferred subject for administration of a construct of the invention, for therapeutic or prophylactic purposes, is a mammal including but is not limited to non human animals (e.g., horses, cows, pigs, dogs, cats, sheep, goats, mice, rats, etc.), and in a preferred embodiment, is a human or non-human primate. In one embodiment, the constructs of the invention are used prophylactically to treat a subject at risk for infection with a pathogen. In another embodiment, the constructs of the invention are used therapeutically to treat a subject with a pathogen infection, harboring a circulating toxin and/or with cancer. In another embodiment, the constructs of the invention are used to immunize a subject against a pathogen or antigen derived therefrom such that the subject develops immunity to the pathogen and/or antigen derived therefrom.

In specific embodiments, an infectious disease and/or symptoms associated therewith is treated or prevented by administration of a construct of the invention.

In one embodiment, construct of the invention is used to treat a subject at high risk of infection. In another embodiment, a construct of the invention is used to treat an immunocompromised patient. In another embodiment, a construct of the invention is used to treat a subject prior to a surgical procedure or implantation of an indwelling device.

In another embodiment, a construct of the invention is used to treat a subject with bacteremia. In another embodiment, a construct of the invention is used to treat a nosocomial infection in a subject. In yet another embodiment, a construct of the invention is used to treat an infection with an antibiotic resistant organism.

In certain embodiments, a construct of the invention is used to induce an immune response in a subject. Induction of such a response in a subject can be measured, e.g., via performance of in vitro assays, e.g., assays designed to detect T- and B-cell activation (e.g., by ELISA or other art-recognized antibody detection assay). The induction of an immune response in a subject induced by a construct of the invention may also be measured in vivo using known techniques, e.g., through assessment of the ability of a treated subject to resist subsequent infection with, e.g., a pathogenic antigen, and/or by proxy via, e.g., measurement of symptoms, morbidity and/or mortality that may be associated with a pathogenic antigen or tumor in an untreated subject.

In one embodiment, a construct of the invention is used as a vaccine to treat a subject at risk of recurring infection or a subject having a recurring infection. In one embodiment, a construct of the invention is administered as a prophylactic vaccine. In another embodiment, a construct of the invention is administered as a therapeutic vaccine (e.g., is administered at some point after infection with a pathogen. Preferably, administration of a construct of the invention results in a protective immune response. A protective immune response can be demonstrated by the ability of immune serum from a protected subject (i.e., a subject that has been treated with a construct of the invention) to passively protect a second subject.

In another embodiment, a construct of the invention can be used as a vaccine adjuvant. In one embodiment, a construct of the invention is administered with an antigen (e.g., a purified bacterial antigen or an attenuated pathogen), optionally in addition to an antigen moiety of the construct.

In an additional embodiment, a construct of the invention is used to clear an antigen from the circulation and/or tissue of a mammal. Measurement of the clearance of an antigen from the circulation can be measured using techniques known in the art. Similarly, measurement of clearance from one or more tissue(s) of a treated mammal may be determined in vivo or in vitro. For example, in vivo determination of the clearance of an antigen from a treated subject may involve, e.g., biopsy or other invasive method of tissue/organ monitoring, or may alternatively involve non-invasive detection of clearance via, e.g., detection of clearance of (optionally labeled) antigen via, e.g., MRI, CAT or other art-recognized imaging method. In vivo assessment of the clearance of antigen from the tissue(s) of a treated mammal may also occur, e.g., via assessment of the ability of a treated subject to resist subsequent infection with, e.g., a pathogenic antigen, and/or by proxy via, e.g., measurement of symptoms, morbidity and/or mortality that may be associated with a pathogenic antigen in an untreated subject. In vitro determination of tissue-clearance of an antigen using a construct of the invention may be performed, e.g., via assessment of biopsy (and/or whole tissues and/or organs) tissues for presence of antigen by any art-recognized means of such detection, including, e.g., antibody-mediated methods of antigen detection, detection of the activity of an antigen, etc. Antigen clearance may be assessed in any and/or all tissue(s) and/or organ(s) of a treated mammal.

In one embodiment, a subject is treated with a construct of the invention and at least one other therapeutic agent designed to treat infection and/or alleviate symptoms. In another embodiment, a subject is treated with a construct of the invention alone.

### VIII. PHARMACEUTICAL FORMULATION AND ADMINISTRATION

The constructs of the invention can be incorporated into pharmaceutical compositions suitable for administration. Such compositions typically comprise a construct and a pharmaceutically acceptable carrier. As used herein the language “pharmaceutically acceptable carrier” includes, e.g., solvents, dispersion media, coatings, antibacterial and antifungal agents, isotonic and absorption delaying agents, and the like, compatible with pharmaceutical administration. The use of such media and agents for pharmaceutically active substances is well known in the art. Supplementary constructs can also be incorporated into the compositions.

A pharmaceutical composition of the invention is formulated to be compatible with its intended route of administration. The preferred route of administration is intravenous. Other examples of routes of administration include intramuscular, parenteral, intradermal, subcutaneous, transdermal (topical), and transmucosal. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). The composition is preferably sterile and should be fluid to the extent that the viscosity is low and the construct is injectable. It is preferably stable under the conditions of manufacture and storage and must be preserved against the contaminating action of microorganisms such as bacteria and fungi.

The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the construct (e.g., one or more constructs) in the required amount in an appropriate solvent with one or a combination of ingredients enumerated above, as required, followed by filtered sterilization. Generally, dispersions are prepared by incorporating the construct into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In one embodiment, the constructs are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811 which is incorporated herein by reference in its entirety.

It is advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of construct calculated to produce the desired immune response and/or therapeutic effect in association with the required pharmaceutical carrier. The specification for the dosage unit forms of the invention are dictated by and directly dependent on the unique characteristics of the specific construct and the particular immune response and/or therapeutic effect to be achieved, and the limitations inherent in the art of compounding such a construct for the prophylaxis and/or treatment of individuals.

The pharmaceutical compositions can be included in a kit, in a container, pack, or dispenser together with instructions for administration.

The construct molecules of the invention may be administered alone or in combination with additional agents to a host. In certain embodiments of the instant invention, such additionally administered agents may include, e.g., antigen, chemotherapeutic agents, antibiotics, antiviral agents, etc.

In specific embodiments of the instant invention (particularly those that feature a construct comprising a CR1-specific binding moiety that is linked to a second moiety comprising an antigen-binding fragment), antigen may additionally be administered to a mammal to which a construct of the invention is administered. Such administration can be performed both to effect a prophylactic and/or therapeutic elevation of an immune response to the additionally administered antigen. The timing of such administration of antigen may precede administration of the construct molecule(s) of the invention, may be performed concurrent with administration of the antigen, or may be performed following administration of the antigen.

The constructs can be delivered via a route determined to be appropriate by one of ordinary skill in the art. For example, the subject constructs ma be administered either subcutaneously, epidermally, intradermally, intramuscularly, intravenous, mucosally (such as nasally, rectally and vaginally), intraperitoneally, orally or combinations thereof. Preferably, the constructs are delivered mucosally. More preferably, the constructs are delivered intranasally, intravaginally, or intrarectally.

Carriers may also be used with the constructs of the invention. Carriers are well known in the art, and include, e.g., thyroglobulin, albumins such as human serum albumin, tetanus toxoid, polyamino acids such as poly L-lysine, poly L-glutamic acid, and the like. The carriers can contain a physiologically tolerable (i.e., acceptable) diluent such as water, or saline, preferably phosphate buffered saline. The constructs of the invention may also be administered with an adjuvant. Adjuvants such as incomplete Freund's adjuvant, aluminum phosphate, aluminum hydroxide, or alum are examples of materials well known in the art.

In another embodiment, polynucleotide compositions of the invention can be used to cause proteins to be synthesized. Such polynucleotides can be delivered using one or more gene vectors, administered via nucleic acid immunization or the like using standard gene delivery protocols. Methods for gene delivery are known in the art. See, e.g., U.S. Pat. Nos. 5,399,346, 5,580,859, 5,589,466. An exemplary replication-deficient gene delivery vehicle that may be used in the practice of the present invention is any of the alphavirus vectors, described in, for example, U.S. Pat. Nos. 6,342,372; 6,329,201 and International Publication WO 01/92552.

The dose for administration of a construct of the invention can be determined by one of ordinary skill in the art upon conducting routine tests. Prior to administration to humans, the efficacy is preferably shown in animal models. Any animal model for production of an immune response known in the art can be used. More particularly, the dose of the construct can be determined based on the immune cell concentration and the number of CR1 epitope sites bound by the constructs of the invention.

As defined herein, a therapeutically effective amount of a construct (i.e., an effective dosage) ranges from about 0.001 to 50 mg/kg body weight, preferably about 0.01 to 5 mg/kg body weight, more preferably about 0.1 to 2 mg/kg body weight, and even more preferably about 0.1 to 1 mg/kg, 0.2 to 1 mg/kg, 0.3 to 1 mg/kg, 0.4 to 1 mg/kg, or 0.5 to 1 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a construct of the invention can include a single treatment or, preferably, can include a series of treatments. In a preferred example, a subject is treated with a construct in the range of between about 0.1 to 5 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a construct, used for treatment may increase or decrease over the course of a particular treatment. Changes in dosage may result and become apparent from the results of diagnostic assays as described herein.

It is understood that appropriate doses of construct agents depend upon a number of factors within the skill of the ordinarily skilled physician, veterinarian, or researcher. The dose(s) of the construct will vary, for example, depending upon the identity, size, and condition of the subject or sample being treated, further depending upon the route by which the composition is to be administered, if applicable, and the effect which the practitioner desires the construct to have upon the immune response.

It is also understood that appropriate doses of constructs depend upon the potency of the construct with respect to the antigenic moiety to which an enhanced immune response occurs. Such appropriate doses may be determined using the assays described herein. When one or more of these constructs is to be administered to an animal (e.g., a human) in order to enhance an immune response to an antigen, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the construct employed, the age, body weight, general health, gender, and diet of the subject, the time of administration, the route of administration, the rate of excretion, any drug combination, and the concentration of antigen to which an enhanced immune response is raised.

### IX. KITS

The invention provides kits containing the constructs, or components necessary to make the constructs, of the invention. Kits containing the pharmaceutical compositions of the invention are also provided.

All references cited herein (including, e.g., books, journal articles, issued patents, and patent applications) are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

### EXAMPLES

### Example 1

### Use of Bispecific Molecules Made Using a Protein A Monoclonal Antibody Against Staphylococcus aureus for Inactivation of the Pathogen

An animal model for lethal challenge with S. aureus will be developed. This model will be used to test the hypothesis that a bispecific molecule made using a protein A Mab will be able to inactivate its target pathogen, S. aureus. The anti-S. aureus Mab to be used is an anti-Protein A Mab (Catalog # P 2921, Sigma Aldrich, St, Louis Mo.). This Mab is likely to be protein A since Protein A is not known to be involved with binding to any surface proteins in animals or humans. A Heteropolymer (HP) made by cross-linking the anti-protein A Mab to the anti-complement receptor type I (CR1) Mab 7G9 will clear the S. aureus to the erythrocyte (E) surface. Based on previous models of HP action, the E:HP:S. aureus complexes will be cleared to the fixed tissue macrophages (Kuppfer cells) in the liver where the immune complex (CR1:HP:S. aureus) will be destroyed. On the other hand, Mab alone will not be as effective in protecting the mice from a lethal S. aureus challenge since (i) Protein A is not involved in tissue invasion and (ii) density of protein A on the surface of the organism is relatively high and all the protein A on the surface may not blocked by the Mab. In contrast to Mab alone, in order for the HP to be effective, there is no need for all the protein A to be bound since a few HPs can tether the microorganism to the E and inactivate the pathogen.

Methods:

The aim of this experiment is to determine the efficacy of bispecific molecules versus Mab at preventing death in CR1 transgenic mice injected with S. aureus. CR1 mice will be injected with either PBS, Mab or bispecific molecule IV followed by S. aureus IV. The groups sizes will be 10 mice/group.

Stock cultures of S. aureus will be prepared, aliquoted and frozen at −80 degrees C. Defrosted bacteria will be titered in advance. On the day of injection, bacteria will be diluted for injection and re-titered. Animals (e.g., mice) will be injected with saline, HPs or Mabs in a total volume of 100 μl IV. One hour later inject S. aureus in a total volume of 100 μL IV. Animals will be monitored for 21 days post-injection or until death. Animals will be monitored twice daily for time to death (TTD) for 21 days. Animals that are moribund will be euthanized. A summary of the experimental design is shown in Table 8.

TABLE 8
Study in an animal model to determine the efficacy of HP versus Mab in
protection against a lethal S. aureus challenge.

| Group ID | # animals | Treatment | Manipulation |
|---|---|---|---|
| 1 | 10 | Saline | S. aureus (˜3xLD100) injected IV |
| | | | after saline |
| 2 | 10 | 20 μg anti-S. aureus | HP injected IV 1 hour prior to S. aureus |
| | | bispecific molecule | (˜3xLD100) injected IV |
| | | (heteropolymer, HP) | |
| | | (7G9 X anti-protein A Mab) | |
| 3 | 10 | 10 μg anti-S. aureus Mab | Mab injected IV 1 hour prior to S. aureus |
| | | (Anti-protein A Mab) | (˜3xLD100) injected IV |
| 4 | 10 | 5 μg anti-S. aureus HP | HP injected IV 1 hour prior to S. aureus |
| | | (7G9 X anti-protein A Mab) | (˜3xLD100) injected IV |
| 5 | 10 | 2.5 μg anti-S. aureus Mab | Mab injected IV 1 hour prior to S. aureus |
| | | (Anti-protein A Mab) | (˜3xLD100) injected IV |
| 6 | 10 | 20 μg control HP | HP injected IV 1 hour prior to S. aureus |
| | | (7G9 X anti-PA Mab14B7) | (˜3xLD100) injected IV |
| 7 | 10 | 10 μg anti-CR1 Mab 7G9 | Mab injected IV 1 hour prior to S. aureus |
| | | | (˜3xLD100) injected IV |

### Example 2

### Administration of Anti-Protein A HPs Before or After Challenge Protects Mice Against Lethal S. Aureus.

Bispecific molecules were made by chemical conjugation of murine MAb 7G9 (anti-DR1) with murine anti-protein A SpA-27 monoclonal antibody (Catalog # P 2921, Sigma Aldrich, St, Louis Mo.). In brief, MAb 7G9 was activated with malemide heterobifunctional crosslinker SMCC. The anti-S aureus MAb was activated with the thiol heterobifunctional crosslinker SATA. The 7G9-malemide and spa-27-sulfhydryl modified proteins were subsequently reacted to produce thio-ether linked bispecific molecule conjugates. Products were purified by size exclusion chromatography using a high resolution Sephacryl 300AE column.

Bispecific molecule mediated delivery of S. aureus to macrophages leads to enhanced bactericidal activity in vitro. Medium, soluble SpA-27, or SpA-27 bispecific molecule were incubated with RBCs and S. aureus. These reaction mixtures were then incubated with RAW 264.7 macrophages for 90 minutes. RBCs were lysed and macrophages were washed. The macrophages were lysed at time 0 hours, 1 hour, 2 hours, 3 hours, 4 hours, and 5 hours and the lysates were plated on agar plates to obtain S. aureus colony counts. The data show that that at 5 hours there was a statistically significant decrease in the colony count of S. aureus internalized by macrophages when incubated with the bispecific molecule. Thus, delivery of S. aureus to macrophages by the bispecific molecule system leads to enhancement of the bactericidal activity of macrophages.

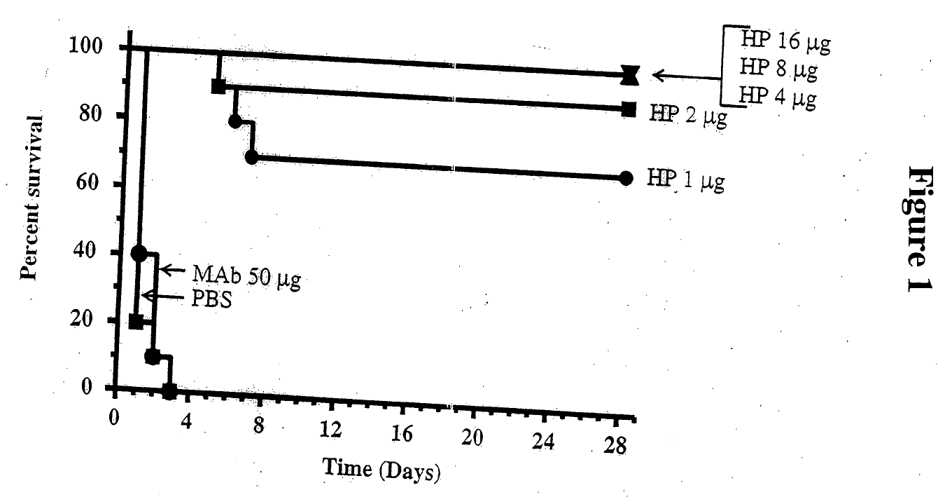

The protective effect on survival of anti-S. aureus Protein A bispecific-treated mice challenged with lethal doses (ten-fold LD₅₀ doses of S. aureus strain MW2) of S. aureus was examined. Ten mice per group were intravenously administered anti-S. aureus Protein A bispecific (1-16 μg/mouse), anti-S. aureus Protein A monoclonal antibody (50 μg/mouse) or PBS. Survival of these groups of mice was observed over a span of 28 days, with complete survival observed for mice administered bispecific molecule doses of 4 μg/mouse, 8 μg/mouse, and 16 μg/mouse (FIG. 1). Anti-S. aureus bispecific molecule administration as low as 1 μg/mouse provided significant protection. Challenge with a genetically distinct strain of S. aureus, 13301, was similarly protective. Bispecific molecules therefore were an effective prophylactic for S. aureus challenge.

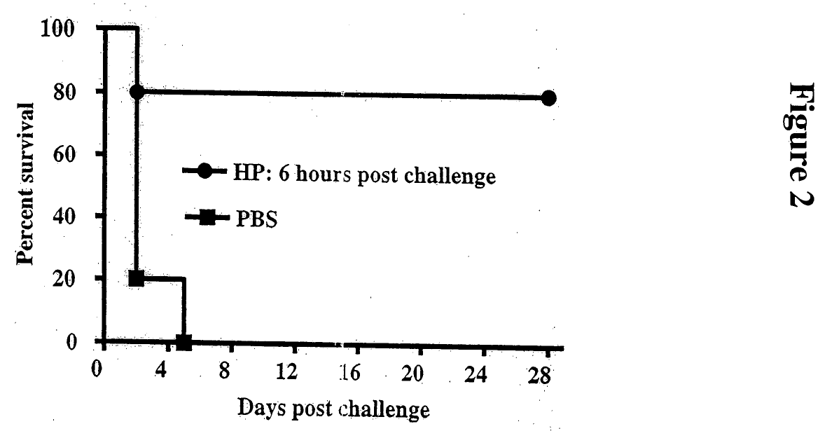

The therapeutic effect on survival of anti-S. aureus Protein A bispecific molecule-treated mice challenged with lethal doses (ten-fold LD₅₀ doses of S. aureus strain MW2) of S. aureus was examined. Ten mice per group were intravenously administered either bispecific molecule or PBS at six hours after mice were challenged with ten-fold LD₅₀ doses of S. aureus strain MW2. A therapeutic effect on survival was observed for the group of mice that were administered the bispecific molecule treatment (FIG. 2).

### Example 3

### Bispecific Molecule Protection Leads to Development of a Robust, Protective Immune Response

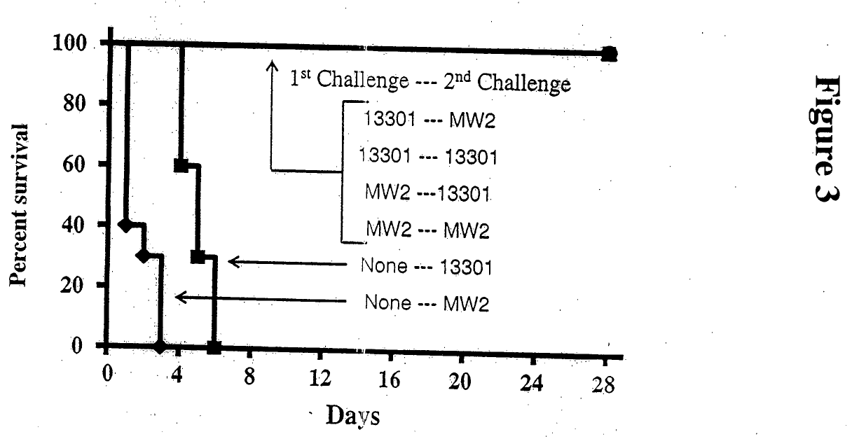

Five mice per group were challenged with bispecific molecule administered 30-45 minutes prior to challenge with S. aureus strains MW2 or 13301 at approximate ten-fold LD₅₀ doses. On day 28, these mice were re-challenged with either of S. aureus strain MW2 or 13301 at approximate ten-fold LD₅₀ doses. Protective effects of HP administration to all bispecific molecule -treated groups of mice persisted through this re-challenge at 28 days (FIG. 3).

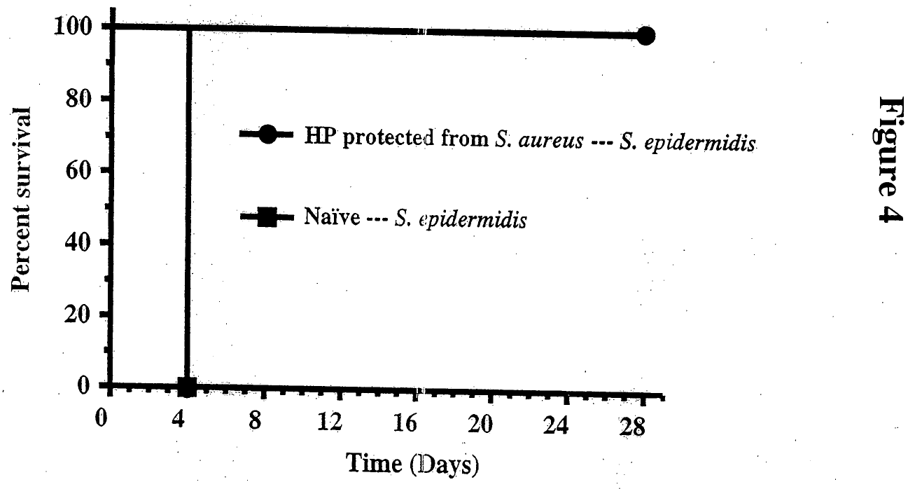

Re-challenge of bispecific molecule -administered mice (5 mice per group) at 28 days with S. epidermidis (strain 10683) following initial challenge by S. aureus was also observed to be protective. Mice re-challenged at day 28 with 10⁸ CFU/mouse (˜ten-fold LD₅₀) of S. epidermidis were all observed to survive this re-challenge (FIG. 4). Bispecific molecules were administered 30-45 minutes prior to initial challenge of these mice. The persistent protective effect of bispecific molecule administration was thus observed to be efficacious against a second challenge with S. epidermidis.

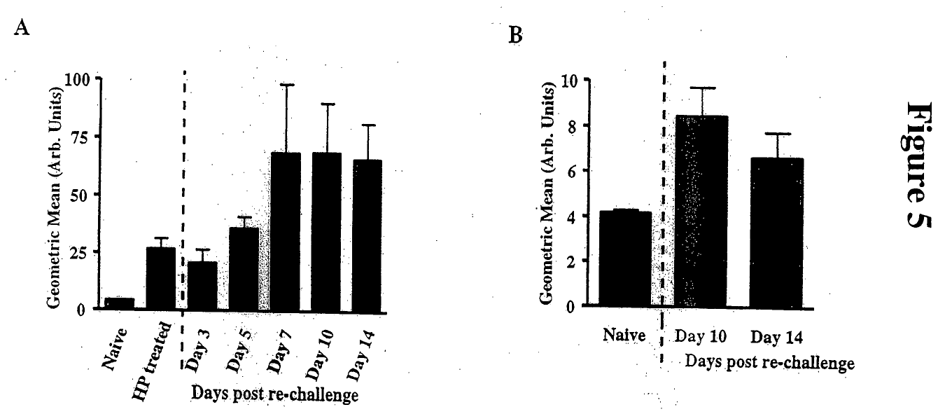

The antibody response of bispecific molecule -treated mice initially challenged with S. aureus was examined. Naïve or bispecific molecule-treated groups. Groups of 5 mice each were challenged with S. aureus, then examined for anti-S. aureus (FIG. 5A) or anti-S. epidermidis (FIG. 5B) antibody response. Bispecific molecule—protected mice generated antibody titers that were 5-10 fold over titers in naive mice by day 28. After re-challenge with S. aureus these mice produced a robust antibody response, 20-25 fold over titers in naive mice by days 7-14 post re-challenge. These antibodies cross-reacted with S. epidermidis. Bispecific molecule-treated mice were thus immune to re-challenge with different strains of S. aureus as well as S. epidermidis.

### Example 4

### Administration of an Anti-Protein A HP Construct Clears S. Aureus From the Blood and Target Organs

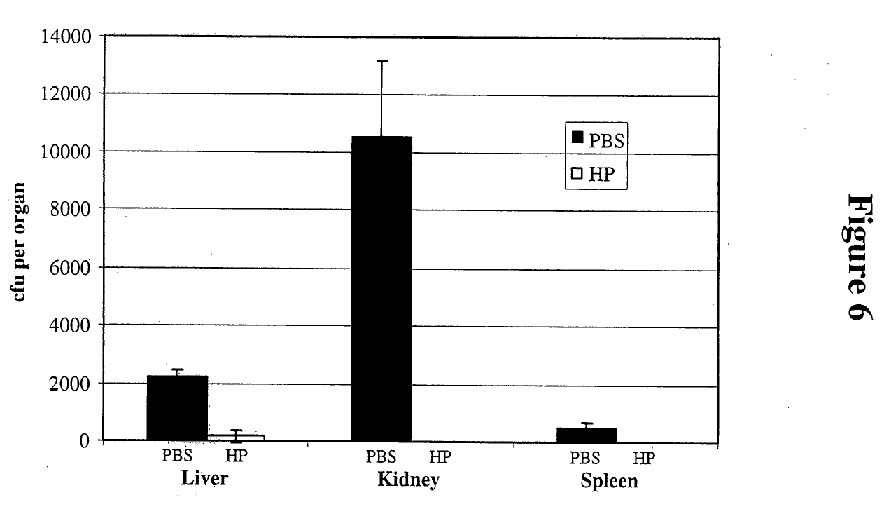

The efficacy of StaphA HP molecules (anti-Protein A HP constructs) at clearing S. aureus from the blood and target organs of mice following injection of S. aureus was examined. Mice were injected with either PBS or StaphA HP (12 μg per mouse) and challenged one hour later with S. aureus strain MW2 (10⁶ cfu per mouse). Initial blood samples collected at a 30 minute time point showed similar bacterial counts for both HP- and PBS-injected mice (5-8×10³). Blood samples were then collected at approximately 84 hours after challenge, and mice were sacrificed following blood collection. All blood samples were plated on agar plates to determine cfu counts. The kidneys, liver and spleen of each mouse were homogenized and plated for cfu counts. Mice administered either StaphA HP molecules or PBS showed no colonies on the plates, indicating that there was no bacteria in the blood in either the StaphA HP or the PBS group. Mice that received StaphA HP molecules exhibited surprisingly robust clearance of S. aureus from the S. aureus target organs of the liver, kidney and spleen (refer to FIG. 6). In all three organs tested, cfu counts were significantly higher in the PBS group compared with the StaphA HP group. There were no detectable counts in the kidney and spleen of mice that had received the StaphA HP, and bacteria counts in the liver were close to background. There was a reduction in cfu counts per organ of up to 4 log units. Thus, StaphA HP molecules were extraordinarily effective at clearing S. aureus from both blood and target organs.

### Example 5

### Effective Treatment of Candida Infection Using a Candida HP Construct

HPs designed to clear systemic Candida albicans infections were generated and tested. Two HPs were generated using two different monoclonal antibodies that recognize the same surface antigen. One of the antibodies, C3.1, is an IgG3, which has previously been used in animal studies, where it was shown to be partially protective. The second MAb, G11.1, is an IgG1 which has previously been shown to be non-protective. Efficacy studies were conducted in mice transgenic for human CR1. Animals (10/group) received PBS, G11.2 HP (20 μg/mouse) or C3.1 HP (20 μg/mouse) one hour prior to a lethal C. albicans challenge (1×10⁶ cfu/mouse) administered iv by the tail vein. As a positive control, one group of mice was treated once daily on study days 1-14 with Ambisome (4 mg/kg), a lipid formulation of Amphotericin. The data were analyzed and presented as Kaplan-Meier survival plots.

Consistent with the C. albicans challenge (1×LD₁₀₀), all mice that had received PBS died between days 4 and 8. All of the positive control Ambisome-treated mice survived to study day 14. Animals treated with Candida HPs showed significant survival and delay in mortality. The C3.1 HP provided 40% survival with deaths occurring between days 3 and 13. This outcome differed significantly from the PBS (P=0.0123) and Ambisome (P=0.0025)-treated mice. Interestingly, these C3.1 HP-treated mice showed a statistically significant difference from G11.1 HP-treated mice (P<0.0001). The G11.1 HP provided 40% survival with deaths occurring on days 8, 10 and 14, an outcome significantly different from the PBS (P=0.0004)-treated mice, but not significantly different from the Ambisome (P=0.0.0700)-treated mice

These data demonstrated that HPs comprising an antibody to a surface epitope on C. albicans are effective therapeutics. The results also showed that the anti-C. albicans antibody does not have to be protective to make an effective drug.

### Example 6

### Vaccination With Constructs that Comprise a CR1 Binding Moiety

Targeting of recombinant protective antigen (PA) to CR1 presents a safe, effective alternative to existing vaccines for anthrax that contain a cell-free filtrate of a non-encapsulated attenuated strain of B. anthracis combined with aluminum hydroxide (Puziss, 1963) or recombinant PA combined with aluminum hydroxide. A molecule that a moiety that binds to CR1 attached to PA or a PA-derived antigen can allow the generation of a protective immune response. Using a mouse model system, mice transgenic for human CR1 are vaccinated with PA (e.g., at dosage of 10 μg/mouse iv) alone, PA (e.g., 10 μg/mouse iv) in the presence of a PA neutralizing monoclonal antibody (MAb; e.g., 5 μg/mouse iv), or a molecule that comprises a moiety that binds to CR1 attached to PA or an antigenic fragment thereof (e.g., 10 μg/mouse iv). Following this vaccination, blood samples are collected from the mice at seven days after initial treatment, and again at seven days after a second boost of the vaccine.

Production of neutralizing antibodies to PA in response to vaccination of TgN hCR1 mice is then assayed. Initially, a PA competitive ELISA is performed, wherein mouse sera are diluted and plated onto PA-coated plates. Following incubation, HRP (horseradish peroxidase) conjugates-14B7 are added and TMB is then added, with the plate read at 450 nm. The data can be reported as the titer required to produce an OD 1.5.

Anthrax lethal toxin (LeTx) neutralization assays are performed as a test of the vaccine. The murine macrophage cell line J774A.1 is used for toxin neutralization assays due to its known sensitivity to rapid cytolysis by LeTx (Little, 1990). Cell viability is measured as signal with the tetrazolium, MTT. A minimal concentration of PA and LF required for complete and/or 50% killing of J774.1 cells is determined (e.g., values likely in the range of 0.16 μg/ml of each protein for complete killing and 0.026 μg/ml of each protein for 50% killing). For toxin neutralization, sera are pre-incubated with LeTx for one hour at 37° C., the mixtures are added to wells containing macrophages, and incubation proceeds for four hours at 37° C. The data can be reported in proportionate relation to a deimmunized anti-PA antibody, Anthim, reported in Mohamed et al. (Mohamed, 2005).

The sera are analyzed for the presence of antibodies that recognize PA in a competitive ELISA, and for the ability of the sera to neutralize lethal toxin in an in vitro assay. Titer and toxin neutralization values are assessed and compared for all vaccination conditions.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for inducing an immune response to an antigen in a mammal comprising administering a molecule effective for clearance of the antigen from the circulation the molecule comprising a first moiety which binds specifically to complement receptor 1 (CR1) linked to a second moiety which binds to the antigen, wherein an immune response to the antigen is induced in the mammal.

2. The method of claim 1, wherein the first moiety comprises an antibody.

3. The method of claim 2, wherein the antibody is an anti-human CR1 antibody.

4. The method of claim 1, wherein the first moiety is an anti-CR1 antibody selected from the group consisting of 7G9, H4, E11, H9, and YZ-1.

5. The method of claim 1, wherein at least one of the first or second moiety comprises an antibody or an antigen binding portion thereof.

6. The method of claim 5, wherein at least one of the first or second moiety is selected from the group consisting of a Fab fragment, a F(ab′)₂ fragment, a single chain antibody, and an scFv molecule.

7. The method of claim 1, wherein the antigen is a pathogenic agent or epitope derived therefrom.

8. The method of claim 7, wherein the pathogenic agent is selected from the group consisting of a virus, a bacterium, a fungus, and a parasite or an epitope derived therefrom.

9. The method of claim 7, wherein the pathogenic agent binds to a receptor on a host cell and the second moiety comprises a soluble form of the receptor.

10. The method of claim 9, wherein the pathogenic agent is a virus and the second moiety comprises a soluble form of a cellular receptor that binds to the virus.

11. The method of claim 1, wherein the second moiety is a small molecule or a drug.

12. The method of claim 8, wherein the pathogenic agent is a fungus and the second moiety comprises amphotericin B.

13. The method of claim 1, wherein the antigen is a toxin or an epitope derived therefrom.

14. The method of claim 1, wherein the antigen is selected from the group consisting of a tumor cell, a tumor cell toxin, an epitope derived from a tumor cell, and an epitope derived from a tumor cell toxin.

15. The method of claim 1, wherein the antigen is a pathogenic protein.

16. The method of claim 6 or 8, wherein the epitope is selected from the group consisting of a protein, a peptide, a carbohydrate, a lipid, a lipopolysaccharide, a polysaccharide, a small molecule, glycoprotein, and a peptidoglycan.

17. The method of claim 1, wherein the first and second moieties are linked via a chemical crosslinker.

18. The method of claim 17, wherein the chemical crosslinker comprises polyethelyene glycol (PEG) as a spacer.

19. The method of claim 17, wherein the first and second moieties are covalently linked.

20. The method of claim 17, wherein the first and second moieties are non-covalently linked.

21. The method of claim 1, wherein the first and second moieties are linked via a genetic fusion.

22. The method of claim 1, wherein the molecule is a heteropolymer.

23. The method of claim 1, wherein the molecule is a bispecific antibody.

24. The method of claim 1, wherein the molecule is a fusion protein.

25. The method of claim 1, wherein the antigen comprises a non-infectious form of a pathogen, a vaccine strain of a pathogen, or epitope derived therefrom, the method further comprising administering the antigen to the mammal.

26. The method of claim 25, wherein the antigen is administered prior to the molecule.

27. The method of claim 25, wherein the antigen is administered with the molecule.

28. The method of claim 27, wherein the antigen is part of the construct.

29. The method of claim 25, wherein the antigen is administered after the molecule.

30. The method of claim 1, wherein at least the first or the second moiety of the molecule is a human antibody.

31. The method of claim 1, wherein at least the first or the second moiety of the molecule is modified to decrease immunogenicity.

32. The method of claim 31, wherein at least one of the first or the second moiety of the molecule comprises an entity selected from the group consisting of a chimeric antibody or antigen binding portion thereof, a humanized antibody or antigen binding portion thereof, and a deimmunized antibody or antigen binding portion thereof.

33. The method of claim 1, wherein the immune response is a protective immune response against the antigen.

34. The method of claim 1, wherein a disease is treated in the mammal.

35. The method of claim 1, wherein a disease is prevented in the mammal.

36. The method of claim 1, wherein an infection is treated in the mammal.

37. The method of claim 36, wherein the infection is a bacterial infection.

38. The method of claim 36, wherein the infection is a viral infection.

39. The method of claim 36, wherein the infection is a fungal infection.

40. The method of claim 36, wherein the infection is a parasitic infection.

41. The method of claim 36, wherein the infection is nosocomial.

42. The method of claim 1, wherein infection is prevented in the mammal.

43. The method of claim 42, wherein the mammal is at risk for recurring infections.

44. The method of claim 42, wherein the mammal has had recurring infections.

45. The method of claim 42, wherein the molecule is administered prior to an invasive medical procedure.

46. The method of claim 45, wherein the procedure is a surgical procedure.

47. A composition for inducing an immune response to an antigen in a subject comprising administering a molecule effective for clearance of the antigen from the circulation the molecule comprising a first moiety which binds specifically to human complement receptor 1 (CR1) linked to a second moiety which binds to the antigen, wherein an immune response to the antigen is induced in the subject.

48. A molecule comprising a first moiety which binds to complement receptor 1 (CR1), linked to a second moiety which binds to Staphylococcus aureus protein A, wherein the molecule is effective for clearance of the antigen from the circulation.

49-68. (canceled)

69. A method of inducing clearance of an antigen from the circulation comprising administering to a mammal having an antigen in its circulation a molecule comprising a first moiety that specifically binds to CR1 and does not substantially bind to CR2 and a second moiety which binds to the antigen such that clearance of the antigen from the circulation is induced.

70-81. (canceled)

82. A construct for inducing an immune response to an antigen in a mammal comprising a first moiety which specifically binds to complement receptor 1 (CR1) linked to a second moiety which binds to the antigen, wherein the construct is not effective for clearing the antigen from the circulation.

83. A construct for inducing an immune response to an antigen in a mammal comprising a first moiety which specifically binds to complement receptor 1 (CR1) linked to a second moiety which comprises the antigen to which an immune response is desired.

84-111. (canceled)

112. A method for inducing an immune response to an antigen in a mammal comprising administering a construct comprising a first moiety which specifically binds to complement receptor 1 (CR1) linked to a second moiety which comprises the antigen to which an immune response is desired to a subject such that in immune response is induced.

113-115. (canceled)

116. The method of claim 112, wherein the antigen is a vaccine strain of a pathogen.

117. A method for inducing an immune response to an antigen in a mammal comprising administering a construct which is not effective for clearing the antigen from the circulation, the construct comprising a first moiety which specifically binds to complement receptor 1 (CR1) linked to a second moiety which binds to the antigen to a subject wherein an immune response to the antigen is induced in the mammal.

118. The method of claim 117, wherein the antigen comprises a non-infectious form of a pathogen, a vaccine strain of a pathogen, or epitope derived therefrom, the method further comprising administering the antigen to the mammal.

119-146. (canceled)

147. A method for clearing an antigen from a tissue of a mammal comprising administering a molecule effective for clearance of the antigen from the tissue, the molecule comprising a first moiety which binds specifically to complement receptor 1 (CR1) linked to a second moiety which binds to the antigen, wherein the antigen is cleared from the tissue of the mammal.

148-194. (canceled)
